SEQUENCE LISTING (8 sequences)

SEQ ID NO: 1 - protein, 222 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGALPETGG

SEQ ID NO: 2 - protein, 450 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 5 aa, synthetic construct
GGGGS

SEQ ID NO: 4 - protein, 10 aa, synthetic construct
GGGGSGGGGS

SEQ ID NO: 5 - protein, 15 aa, synthetic construct
GGGGSGGGGSGGGGS

SEQ ID NO: 6 - protein, 4 aa, synthetic construct
GGFG

SEQ ID NO: 7 - protein, 4 aa, synthetic construct
GFLG

SEQ ID NO: 8 - protein, 4 aa, synthetic construct
ALAL

(19) United States
(12) Patent Application Publication
(10) Pub. No.: US 2026/0284211 A1
(43) Pub. Date: Sep. 24, 2026

(54) DISACCHARIDE LINKER, LINKER-PAYLOAD CONTAINING THE SAME AND GLYCAN CHAIN-REMODELED ANTIBODY-DRUG CONJUGATE, PREPARATION METHOD AND USES THEREOF

(71) Applicant: GENEQUANTUM HEALTHCARE (SUZHOU) CO., LTD., Suzhou (CN)

(72) Inventors: Meijun XIONG, Suzhou (CN); Zhaoxiong CAI, Suzhou (CN); Yajun SUN, Suzhou (CN); Gang QIN, Suzhou (CN); Lili SHI, Suzhou (CN)

(21) Appl. No.: 19/470,950

(22) PCT Filed: Mar. 29, 2024
(86) PCT No.: PCT/CN2024/084808

§ 371 Date: Sep. 30, 2025

(30) Foreign Application Priority Data
Mar. 30, 2023 (WO) PCT/CN2023/085098
Dec. 1, 2023 (WO) PCT/CN2023/135857

(51) Int. Cl. A61K 47/68; A61P 35/00

(52) U.S. Cl. CPC A61K 47/6803; A61K 47/6855; A61K 47/6889; A61P 35/00

(57) ABSTRACT

A linker-payload compound containing disaccharide group and Eribulin, wherein the disaccharide group is connected to the rest of the compound through amide bond. Provided also relates to an antibody-drug conjugate (ADC) containing the linker-payload compound, wherein the disaccharide group in the linker-payload compound is used to remodel the glycan chain in the antibody, and a preparation method and uses described above.

### TECHNICAL FIELD

The disclosure relates to a disaccharide linker. The present disclosure also relates to a linker-payload compound containing disaccharide group and Eribulin, wherein the disaccharide group is connected to the rest of the compound through amide bond. The disclosure also relates to an antibody-drug conjugate (ADC) containing the linker-payload compound, wherein the disaccharide group in the linker-payload compound is used to remodel the glycan chain in the antibody. The disclosure also relates to a preparation method and uses thereof.

### BACKGROUND

Cancer is one of the main causes of human death. About one-sixth of the deaths in the world are related to cancer every year. In 2017, there were 24.5 million new cancer cases and 9.6 million cancer deaths worldwide. The main methods of cancer treatment are surgery, radiotherapy and drug therapy. With the development and application of these therapies, the living conditions of cancer patients have been greatly improved. Drug therapy for cancer has experienced three generations of development: chemotherapy, targeted therapy and immunotherapy. Chemotherapy plays an important role in cancer treatment, but it also has great non-therapeutic side effects, which can also kill a large number of normal cells when killing cancer cells. Targeted therapy reduces the serious side effects of traditional chemotherapy to a certain extent. The therapy mainly uses small molecular targeting drugs (mainly tyrosine kinase inhibitors at present) or monoclonal antibodies to proteins involved in the growth and survival of tumor cells (i.e., targets) to kill tumor cells and tissues. Antibody-drug conjugate (ADC) is a new class of targeted drugs, which is a new drug molecule formed by connecting small molecules with high activity to monoclonal antibodies through chemical linkage. ADC drugs have both the high activity of small molecule drugs and the high specificity and targeting of antibody drugs, which can reduce the non-therapeutic side effects of small molecule toxins on important tissues and organs such as liver, kidney, nerve and heart to some extent, and overcome the limitation of antibody therapy on solid tumors, and it has become one of the hot directions in the research and development of anti-tumor drugs.

The development of ADC drugs involves a systematic engineering in which four factors, such as monoclonal antibody, bioactive small molecule, linker and conjugating mode, are carefully regulated, among which the conjugating mode greatly affects the drug-related properties such as drug stability, metabolic consistency and quality control (Tsuchikama K, An Z. Antibody-drug conjugates: recent advances in conjugation and linker chemistries. Protein Cell 2018, 9, 33-46). The ADC drugs currently on the market and in clinical stage mainly adopt random conjugating based on lysine or cysteine residues, and often get highly heterogeneous mixtures with random conjugating sites and uneven drug/antibody ratio (DAR). It is prone to problems in process stability, quality control, drug stability, metabolic consistency, safety, and other aspects.

In order to overcome the above problems, in recent years, the industry has developed a new generation of site-specific conjugating technologies, such as introducing specific amino acids based on antibody engineering, site-specific conjugating based on transpeptidase catalysis, affinity-mediated site-specific conjugating and site-specific conjugating of the antibody glycan chain catalyzed by enzymes, which has solved the limitation of traditional conjugating heterogeneity to some extent. However, these technologies still have some limitations, such as the partial conjugating technology needs additional antibody modification or engineering transformation, the screening and synthesis of affinity ligands are complicated, the conjugating conditions are not mild and green enough, the process flow is complicated, and the application scope of substrates is limited (Walsh S J, Bargh J D, Dannheim F M, et al. Site-selective modification strategies in antibody-drug conjugates. Chem. Soc. Rev. 2021, 50, 1305). The site-specific conjugating of the antibody glycan chain catalyzed by enzymes technology uses glycosyltransferase or endoglycosidase to modify the antibody glycan chain and introduce effector molecules, which can realize the site-specific quantitative connection of small molecular drugs to antibodies without additional antibody engineering transformation.

At present, most of the toxins used in ADCs are microtubule-damaging drugs and DNA-damaging drugs, such as calendulin, Mertansine, pyrrolobenzodiazepines, calicheamicins, camptothecins and so on. Eribulin is the first soft spongosine type microtubule kinetic inhibitor with a novel mechanism of action. Eribulin is thought to prevent cell division by inhibiting the growth period of microtubule dynamics. Different from other antimitotic agents (paclitaxel, vinblastine, etc.), the blocking effect of Eribulin on mitosis is irreversible, ultimately leading to cell death. Eribulin can be used as monotherapy, and its efficacy and toxicity have been clinically verified. It is the first drug that shows survival benefits in recurrent/refractory soft tissue sarcoma. Patients are usually resistant to chemotherapy drugs, and the emergence of Eribulin, a novel type of non-taxanes microtubule inhibitor, will provide new possibilities for the treatment of these patients.

At present, the Eribulin ADCs represented by MORAb-202 (Eisai), show better anti-tumor activity in clinic compared with ADCs containing MMAE, MMAF or DM1. However, all these Eribulin ADCs adopt chemical random conjugation, and the low clinical dose and the narrow treatment window limit the further development of Eribulin ADCs.

The present disclosure aims at developing Eribulin ADCs using a novel enzymatic site-specific conjugation technology in which the antibody glycan chain is remodeled to overcome the problems existing in current Eribulin ADCs.

### SUMMARY

The disclosure provides a novel disaccharide linker, and a preparation method and uses thereof. The present disclosure also provides a linker-payload compound containing disaccharide group and Eribulin, wherein the disaccharide group is connected to the rest of the compound through amide bond. Specifically, the disclosure provides a linker-payload compound, which has the structure of formula (I):

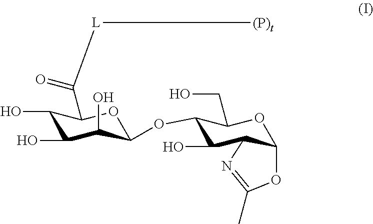

wherein
L is a connecting moiety comprising —NH—, and L is directly connected with the carbonyl group in the disaccharide moiety via the —NH—, wherein when L is a linear connecting moiety, it is connected with one P moiety, and t is 1, or when L is a branched connecting moiety, each of the branches is connected with one P moiety at most, and t is an integer greater than 1 (for example, 2, 3, 4, 5, 6, 7, 8, 9 or 10);
P is a payload with the structure of

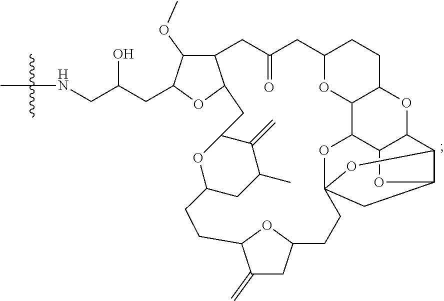 [SMILES: *NCC(O)CC1OC2CC3OC(CCC4OC(CCC56CC7OC8C(OC9CCC(CC(=O)CC2C1OC)OC9C8O5)C7O6)CC4=C)CC(C)C3=C]

preferably, P is Eribulin with the structure of

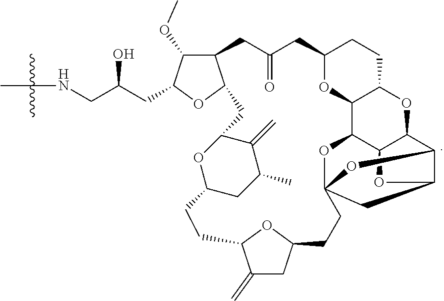 [SMILES: *NC[C@@H](O)C[C@H]1O[C@H]2C[C@H]3O[C@@H](CC[C@@H]4O[C@@H](CC[C@@]56C[C@H]7O[C@H]8[C@@H](O5)[C@H]5O[C@H](CC[C@@H]5O[C@H]8[C@H]7O6)CC(=O)C[C@@H]2[C@H]1OC)CC4=C)C[C@@H](C)C3=C]

The disclosure also relates to an antibody-drug conjugate (ADC) containing the linker-payload compound, wherein the disaccharide group in the linker-payload compound is used to remodel the glycan chain in the antibody. Specifically, the disclosure provides an antibody-drug conjugate having the formula (II) based on the site-specific conjugating of the N-glycosylation site in Fc region of the antibody:

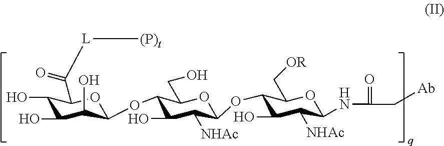

wherein
R is hydrogen or α-L-fucosyl;
q is 1 or 2;
Ab is an antibody or antigen-binding fragment;
L is a connecting moiety comprising —NH—, and L is directly connected with the carbonyl group in the disaccharide moiety via the —NH—, wherein when L is a linear connecting moiety, it is connected with one P moiety, and t is 1; or when L is a branched connecting moiety, each of the branches is connected with one P moiety at most, and t is an integer greater than 1 (for example, 2, 3, 4, 5, 6, 7, 8, 9 or 10);
P is a payload with the structure of

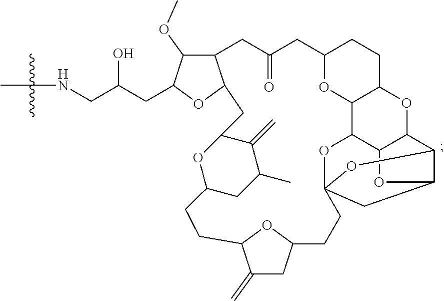 [SMILES: *NCC(O)CC1OC2CC3OC(CCC4OC(CCC56CC7OC8C(OC9CCC(CC(=O)CC2C1OC)OC9C8O5)C7O6)CC4=C)CC(C)C3=C]

preferably, P is Eribulin with the structure of

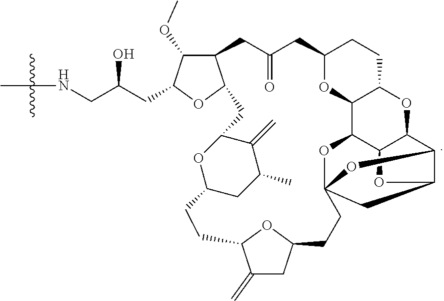 [SMILES: *NC[C@@H](O)C[C@H]1O[C@H]2C[C@H]3O[C@@H](CC[C@@H]4O[C@@H](CC[C@@]56C[C@H]7O[C@H]8[C@@H](O5)[C@H]5O[C@H](CC[C@@H]5O[C@H]8[C@H]7O6)CC(=O)C[C@@H]2[C@H]1OC)CC4=C)C[C@@H](C)C3=C]

The disclosure also relates to the linker-payload compound containing a disaccharide group and a preparation method and uses of antibody-drug conjugate containing the linker-payload compound.

### BRIEF DESCRIPTION OF THE DRAWINGS

FIG. 1 shows the inhibitory effects of the ADCs on SK-BR-3 tumor cells.

FIG. 2 shows the inhibitory effects of the ADCs on NCI-N87 tumor cells.

FIG. 3 shows the inhibitory effects of the ADCs on MDA-MB-468 tumor cells.

FIG. 4 shows the inhibitory effect of ADC-1 on the NCI-N87 human gastric cancer cells in the model of transplatable subcutaneously tumors in mouse.

FIG. 5 shows the influence of ADC-1 on the body weight in the model of transplatable subcutaneously tumors in mouse.

FIG. 6 shows the inhibitory effect of ADCs on the Capan-1 human pancreatic cancer cells in the model of transplatable subcutaneously tumors in mouse.

FIG. 7 shows the influence of ADCs on the body weight in the model of transplatable subcutaneously tumors in mouse.

### DETAILED DESCRIPTION

### General Definition

Unless otherwise defined hereinafter, all technical and scientific terms used herein have the same meaning as commonly understood by those skilled in the art. The techniques used herein refer to those that are generally understood in the art, including the variants and equivalent substitutions that are obvious to those skilled in the art. While the following terms are believed to be readily comprehensible by those skilled in the art, the following definitions are set forth to better illustrate the present disclosure. When a trade name is present herein, it refers to the corresponding commodity or the active ingredient thereof. All patents, published patent applications and publications cited herein are hereby incorporated by reference.

When a certain amount, concentration, or other value or parameter is set forth in the form of a range, a preferred range, or a preferred upper limit or a preferred lower limit, it should be understood that it is equivalent to specifically revealing any range formed by combining any upper limit or preferred value with any lower limit or preferred value, regardless of whether the said range is explicitly recited. Unless otherwise stated, the numerical ranges listed herein are intended to include the endpoints of the range and all integers and fractions (decimals) within the range. For example, the expression “i is an integer of 1 to 20” means that i is any integer of 1 to 20, for example, i can be 1, 2, 3, 4, 5, 6, 7, 8, 9, 10, 11, 12, 13, 14, 15, 16, 17, 18, 19 or 20. Other similar expressions such as j should also be understood in a similar manner.

Unless the context clearly dictates otherwise, singular forms like “a” and “the” include the plural forms. The expression “one or more” or “at least one” may mean 1, 2, 3, 4, 5, 6, 7, 8, 9 or more.

The terms “about” and “approximately”, when used in connection with a numerical variable, generally mean that the value of the variable and all values of the variable are within experimental error (for example, within a 95% confidence interval for the mean) or within ±10% of a specified value, or a wider range.

The term “optional” or “optionally” means the event described subsequent thereto may, but not necessarily happen, and the description includes the cases wherein said event or circumstance happens or does not happen.

The expressions “comprising”, “including”, “containing” and “having” are open-ended, and do not exclude additional unrecited elements, steps, or ingredients. The expression “consisting of” excludes any element, step, or ingredient not designated. The expression “consisting essentially of” means that the scope is limited to the designated elements, steps or ingredients, plus elements, steps or ingredients that are optionally present that do not substantially affect the essential and novel characteristics of the claimed subject matter. It should be understood that the expression “comprise” encompasses the expressions “substantially consist of” and “consist of”.

The term “targeting molecule” refers to a molecule that has an affinity for a particular target (e.g., a receptor, a cell surface protein, a cytokine, a tumor specific antigen, etc.). A targeting molecule can deliver the payload to a specific site in vivo through targeted delivery. A targeting molecule can recognize one or more targets. The specific target site is defined by the target it recognizes. For example, a targeting molecule that targets a receptor can deliver a cytotoxin to a site containing a large number of the receptors. Examples of targeting molecules include, but are not limited to antibody, antibody fragments, binding proteins for a given antigen, antibody mimics, scaffold proteins having affinity for a given target, ligands, and the like. In an embodiment, the target recognized by the targeting molecule is ErbB2/HER2.

HER2 refers to human epidermal growth factor receptor-2, which belongs to the epidermal growth factor (EGFR) receptor tyrosine kinase family. In the present application, the terms ErbB2 and HER2 have the same meaning and can be used interchangeably.

As used herein, the term “antibody” is used in a broad way and particularly includes an intact monoclonal antibody, a polyclonal antibody, a monospecific antibody, a multispecific antibody (e.g., a bispecific antibody), and an antibody fragment, as long as they have the desired biological activity. The antibody may be of any subtype (such as IgG, IgE, IgM, IgD, and IgA) or subclass, and may be derived from any suitable species. In some embodiments, the antibody is of human or murine origin. The antibody may also be a fully human antibody, humanized antibody or chimeric antibody prepared by recombinant methods.

Monoclonal antibodies are used herein to refer to antibodies obtained from a substantially homogeneous population of antibodies, i.e., the individual antibodies constituting the population are identical except for a small number of possible natural mutations. Monoclonal antibodies are highly specific for a single antigenic site, multiple antigenic sites or different epitopes of the same antigen. The word “monoclonal” refers to that the characteristics of the antibody are derived from a substantially homogeneous population of antibodies and are not to be construed as requiring some particular methods to produce the antibody.

An intact antibody or full-length antibody essentially comprises the antigen-binding variable region(s) as well as the light chain constant region(s) (C_{L}) and heavy chain constant region(s) (C_{H}), which could include C_{H}1, C_{H}2, C_{H}3 and/or C_{H}4, depending on the subtype of the antibody. An antigen-biding variable region (also known as a fragment variable region, Fv fragment) typically comprises a light chain variable region (V_{L}) and a heavy chain variable region (V_{H}). A constant region can be a constant region with a native sequence (such as a constant region with a human native sequence) or an amino acid sequence variant thereof. The variable region recognizes and interacts with the target antigen. The constant region can be recognized by and interacts with the immune system.

An antibody fragment may comprise a portion of an intact antibody, preferably its antigen binding region or variable region. Examples of antibody fragments include Fab, Fab′, F(ab′)2, Fd fragment consisting of V_{H} and C_{H}1 domains, Fv fragment, single-domain antibody (dAb) fragment, and isolated complementarity determining region (CDR). The Fab fragment is an antibody fragment obtained by papain digestion of a full-length immunoglobulin, or a fragment having the same structure produced by, for example, recombinant expression. A Fab fragment comprises a light chain (comprising a V_{L} and a C_{L}) and another chain, wherein the said other chain comprises a variable domain of the heavy chain (V_{H}) and a constant region domain of the heavy chain (C_{H}1). The F(ab′)2 fragment is an antibody fragment obtained by pepsin digestion of an immunoglobulin at pH 4.0-4.5, or a fragment having the same structure produced by, for example, recombinant expression. The F(ab′)2 fragment essentially comprises two Fab fragments, wherein each heavy chain portion comprises a few additional amino acids, including the cysteines that form disulfide bonds connecting the two fragments. A Fab′ fragment is a fragment comprising one half of a F(ab′)2 fragment (one heavy chain and one light chain). The antibody fragment may comprise a plurality of chains joined together, for example, via a disulfide bond and/or via a peptide linker. Examples of antibody fragments also include single-chain Fv (scFv), Fv, dsFv, diabody, Fd and Fd′ fragments, and other fragments, including modified fragments. An antibody fragment typically comprises at least or about 50 amino acids, and typically at least or about 200 amino acids. An antigen-binding fragment can include any antibody fragment that, when inserted into an antibody framework (e.g., by substitution of the corresponding region), can result in an antibody that immunospecifically binds to the antigen.

In particular, the antibody-drug conjugate of the present disclosure is based on any natural N-glycosylation modified site contained in antibody Fc region to carry out site-specific conjugating, and the molecules containing glycan chains in antibody Fc region (including but not limited to antibody/double antibody/Fc fusion protein/single chain antibody/nano antibody, etc.) are prepared by one-step method with the linker-payload compound containing oligosaccharide of the present disclosure. Therefore, the antibody of the present disclosure is not particularly limited, as long as the Fe region contains glycan chains, and it can be a natural antibody.

In addition, the antibodies according to the present disclosure can be also prepared using techniques well known in the art, such as the following techniques or a combination thereof: recombinant/genetic engineering techniques, phage display techniques, synthetic techniques, or other techniques known in the art. For example, a genetically engineered recombinant antibody can be expressed by a suitable culture system (e.g., E. coli or mammalian cells). The genetic engineering can refer to, for example, the introduction of a ligase-specific recognition sequence at its terminals.

As used herein, the term “targeting molecule-drug conjugate” is referred to as “conjugate”.

A small molecule compound refers to a molecule with a size comparable to that of an organic molecule commonly used in medicine. The term does not encompass biological macromolecules (e.g., proteins, nucleic acids, etc.), but encompasses low molecular weight peptides or derivatives thereof, such as dipeptides, tripeptides, tetrapeptides, pentapeptides, and the like. Typically, the molecular weight of the small molecule compound can be, for example, about 100 to about 2000 Da, about 200 to about 1000 Da, about 200 to about 900 Da, about 200 to about 800 Da, about 200 to about 700 Da, about 200 to about 600 Da, about 200 to about 500 Da.

Cytotoxin refers to a substance that inhibits or prevents the expression activity of a cell, cellular function, and/or causes destruction of cells. The cytotoxins currently used in ADCs are more toxic than chemotherapeutic drugs. Examples of cytotoxins include, but are not limited to, drugs that target the following targets: microtubule cytoskeleton, DNA, RNA, kinesin-mediated protein transport, regulation of apoptosis. In an embodiment, the cytotoxin is Eribulin.

A spacer is a structure that is located between different structural modules and can spatially separate the structural modules. The definition of spacer is not limited by whether it has a certain function or whether it can be cleaved or degraded in vivo. Examples of spacers include but are not limited to amino acids and non-amino acid structures, wherein non-amino acid structures can be, but are not limited to, amino acid derivatives or analogues. “Spacer sequence” refers to an amino acid sequence serving as a spacer, and examples thereof include but are not limited to a single amino acid, a sequence containing a plurality of amino acids, for example, a sequence containing two amino acids such as GA, etc., or, for example, GGGGS (SEQ ID No. 3), GGGGSGGGGS (SEQ ID No. 4), GGGGSGGGGSGGGGS (SEQ ID No. 5), etc. Self-immolative spacers (e.g., the self-immolative spacer Sp1) are covalent assemblies, which makes two chemical bonds in the precursor cleave successively after the protective part is activated: the protective part (for example, cleavable sequence) is removed after activation, which leads to a cascade decomposition reaction, resulting in the release of smaller molecules in sequence. Examples of self-immolative spacers include but not limited to PABC (p-aminobenzyloxycarbonyl), acetal, heteroacetal or a combination thereof.

As used herein, the term “amino acid” includes “natural amino acid” and “unnatural amino acid”.

The term “natural amino acids” refers to amino acids, which are protein-forming amino acids, including twenty common amino acids (alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine and valine) and less common Selenocysteine and pyrrolysine.

As use herein, that term “unnatural amino acid” refers to an amino acid, which is not a protein constituent amino acid. Specifically, the term refers to an amino acid that is not a natural amino acid as defined above.

The term “alkyl” refers to a straight or branched saturated aliphatic hydrocarbon group consisting of carbon atoms and hydrogen atoms, which is connected to the rest of the molecule through a single bond. The alkyl group may contain 1 to 20 carbon atoms, referring to C₁-C₂₀ alkyl group, for example, C₁-C₄ alkyl group, C₁-C₃ alkyl group, C₁-C₂ alkyl, C₃ alkyl, C₄ alkyl, C₃-C₆ alkyl. Non-limiting examples of alkyl groups include but are not limited to methyl, ethyl, propyl, butyl, pentyl, hexyl, isopropyl, isobutyl, sec-butyl, tert-butyl, isopentyl, 2-methylbutyl, 1-methylbutyl, 1-ethylpropyl, 1,2-dimethylpropyl, neopentyl, 1,1-dimethylpropyl, 4-methylpentyl, 3-methylpentyl, 2-methylpentyl, 1-methylpentyl, 2-ethylbutyl, 1-ethylbutyl, 3,3-dimethylbutyl, 2,2-dimethyl butyl, 1,1-dimethylbutyl, 2,3-dimethylbutyl, 1,3-dimethylbutyl or 1,2-dimethylbutyl, or their isomers. A bivalent radical refers to a group obtained from the corresponding monovalent radical by removing one hydrogen atom from a carbon atom with free valence electron(s). A bivalent radical has two connecting sites which are connected to the rest of the molecule. For example, an “alkylene” or an “alkylidene” refers to a saturated divalent hydrocarbon group, either straight or branched. Examples of alkylene groups include but are not limited to methylene (—CH₂—), ethylene (—C₂H₄—), propylene (—C₃HO—), butylene (—C₄H₈—), pentylene (—C₅H₁₀—), hexylene (—C₆H₁₂—), 1-methylethylene (—CH(CH₃)CH₂—), 2-methylethylene (—CH₂CH(CH₃)—), methylpropylene, ethylpropylene, and the like.

When “substitution” is mentioned herein, unless otherwise specified, the relevant substituents are selected from alkyl, halogen, amino, monoalkylamino, dialkylamino, nitro, cyano, formyl, alkylcarbonyl, carboxyl, alkyloxycarbonyl, alkylcarbonyloxy, aminocarbonyl, monoalkylaminocarbonyl, dialkylaminocarbonyl, formylamino, alkylcarbonylamino, formyl (monoalkyl) amino or alkylcarbonyl (monoalkyl) amino.

As used herein, when a group is combined with another group, the connection of the groups may be linear or branched, provided that a chemically stable structure is formed. The structure formed by such a combination can be connected to other moieties of the molecule via any suitable atom in the structure, preferably via a designated chemical bond.

When multiple identical letters representing chemical groups appear in the same chemical structural formula, they are selected independently and are not necessarily the same. For example, LKa-L²-L¹-Eribulin in the formula (II) comprises multiple L¹ and L², and they are independent of each other and not necessarily the same.

As used herein, the expressions “antibody-conjugated drug” and “antibody-drug conjugate” has the same meaning.

### Linker-Payload Compound

In a first aspect, the disclosure provides a linker-payload compound, which has the structure of formula (I):

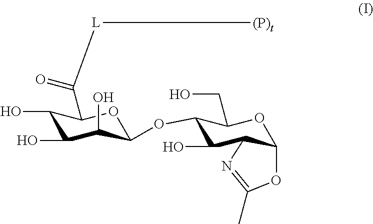

wherein
L is a connecting moiety comprising —NH—, and L is directly connected with the carbonyl group in the disaccharide moiety via the —NH—, wherein when L is a linear connecting moiety, it is connected with one P moiety, and t is 1; or when L is a branched connecting moiety, each of the branches is connected with one P moiety at most, and t is an integer greater than 1 (for example, 2, 3, 4, 5, 6, 7, 8, 9 or 10);
P is a payload with the structure of

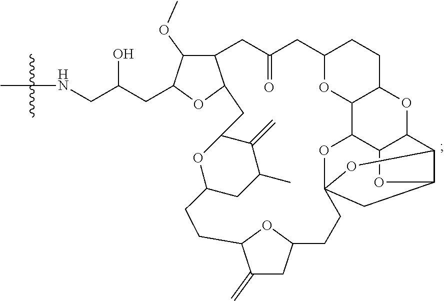 [SMILES: *NCC(O)CC1OC2CC3OC(CCC4OC(CCC56CC7OC8C(OC9CCC(CC(=O)CC2C1OC)OC9C8O5)C7O6)CC4=C)CC(C)C3=C]

preferably, P is Eribulin with the structure of

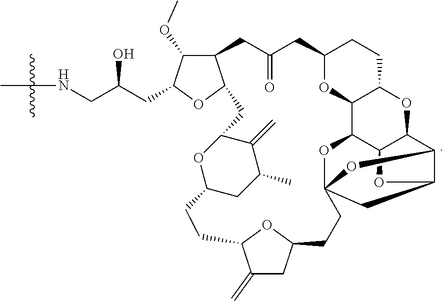 [SMILES: *NC[C@@H](O)C[C@H]1O[C@H]2C[C@H]3O[C@@H](CC[C@@H]4O[C@@H](CC[C@@]56C[C@H]7O[C@H]8[C@@H](O5)[C@H]5O[C@H](CC[C@@H]5O[C@H]8[C@H]7O6)CC(=O)C[C@@H]2[C@H]1OC)CC4=C)C[C@@H](C)C3=C]

In an embodiment, -L- is -L²-L¹- and t is 1, and the compound of formula (I) has the structure of formula (I-1):

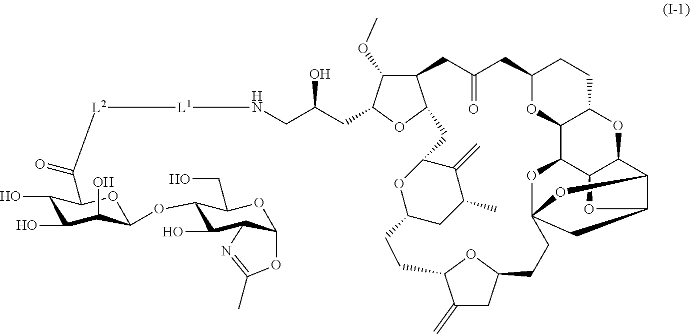

wherein
L¹ comprises a cleavable sequence containing an amino acid sequence that is enzymatically cleavable, wherein the amino acid sequence contains 1, 2, 3, 4, 5, 6, 7, 8, 9 or 10 amino acids;
L² comprises an amino acid residue sequence of -*(AA)ₙ**-, wherein n is an integer of 1-100, AA is an amino acid residue independently at each occurrence, * represents the N-terminal of the corresponding amino acid, ** represents the C-terminal of the corresponding amino acid, and —(C₂H₄—O)ₘ—(CH₂)ₚ— is optionally present between the amino group and the α-carbon of an amino acid, wherein m is 1, 2, 3, 4, 5, 6, 7, 8, 9 or 10; p is 0, 1, 2 or 3, and the * terminal forms an amide bond with the carbonyl group in the disaccharide moiety.

In another embodiment, the compound of formula (I) has the structure of formula (1-2):

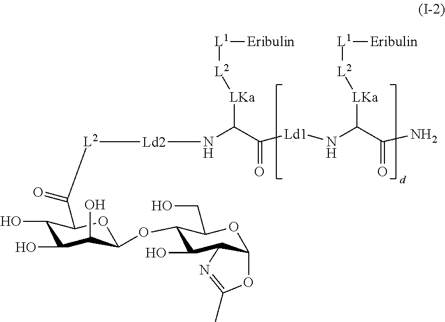

wherein,
Ld2 is independently a bond, or a natural amino acid or an oligomeric natural amino acid with a polymerization degree of 2-10 (i.e. 2, 3, 4, 5, 6, 7, 8, 9 or 10), wherein the side chain of the natural amino acid or the side chain of the oligomeric natural amino acid is independently unsubstituted or substituted by —CO-(PEG)ⱼ-R¹¹;
Ld1 is independently a bond or —NH-(PEG)ᵢ-(CO)—;
—(PEG)ᵢ- and -(PEG)ⱼ- are each independently a PEG fragment, wherein PEG represents —(O—C₂H₄)— or —(C₂H₄—O)—, with an optional C₁₋₁₀ alkylene at one end;
each LKa is independently selected from

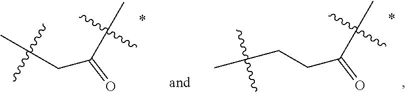 [SMILES: *CC(*)=O ; *CCC(*)=O]

where * represents the connection site with L²;
each L² comprises an amino acid residue sequence of -*(AA)ₙ**-, wherein n is an integer of 1-100, AA is an amino acid residue independently at each occurrence, * represents the N-terminal of the corresponding amino acid, ** represents the C-terminal of the corresponding amino acid, and —(C₂H₄—O)ₘ—(CH₂)ₚ— is optionally present between the amino group and the α-carbon of an amino acid, wherein m is 1, 2, 3, 4, 5, 6, 7, 8, 9 or 10; p is 0, 1, 2 or 3, and the * terminal forms an amide bond with the carbonyl group in the disaccharide moiety; preferably, -Gly-Gly-Gly-;
L¹ comprises a cleavable sequence containing an amino acid sequence that is enzymatically cleavable, wherein the amino acid sequence contains 1, 2, 3, 4, 5, 6, 7, 8, 9 or 10 amino acids;
R¹¹ is C₁₋₁₀ alkyl;
d is 0, 1, 2, 3, 4, 5 or 6;
each i is independently an integer of 1-100 (for example, 1, 2, 3, 4, 5, 6, 7, 8, 9 or 10), preferably 1 to 20; preferably each i is independently an integer of 1 to 12; more preferably 2 to 8; particularly 4;
each j is independently an integer of 1-100 (for example, 1, 2, 3, 4, 5, 6, 7, 8, 9, 10, 11 or 12), preferably 1 to 20; preferably each j is independently an integer of 1 to 12; more preferably 8 to 12; particularly 8 or 12.

In an embodiment, L² comprises an amino acid residue sequence of -*(AA)ₙ**-, wherein n is an integer of 1-100, AA is an amino acid residue independently at each occurrence, * represents the N-terminal of the corresponding amino acid, ** represents the C-terminal of the corresponding amino acid, and —(C₂H₄—O)ₘ—(CH₂)ₚ— is optionally present between the amino group and the α-carbon of an amino acid, wherein m is 1, 2, 3, 4, 5, 6, 7, 8, 9 or 10; p is 0, 1, 2 or 3, and the * terminal forms an amide bond with the carbonyl group in the disaccharide moiety. In an embodiment, L² comprises an amino acid residue sequence of -*(AA)ₙ**-, wherein n is an integer of 1-100, AA is an amino acid residue independently at each occurrence, * represents the N-terminal of the corresponding amino acid, ** represents the C-terminal of the corresponding amino acid, and —(C₂H₄—O)ₘ—(CH₂)ₚ— is optionally present between the amino group and the α-carbon of an amino acid, wherein m is 2; p is 2.

In an embodiment, L² comprises an amino acid residue sequence of -*(AA)ₙ**-, wherein n is an integer of 1-100, AA is an amino acid residue independently at each occurrence, * represents the N-terminal of the corresponding amino acid, ** represents the C-terminal of the corresponding amino acid, and —(C₂H₄—O)ₘ—(CH₂)ₚ— is optionally present between the amino group and the α-carbon of an amino acid, wherein m is 4; p is 2. In an embodiment, AA is independently any one of Phe, Lys, Gly, Ala, Leu, Asn, Val, Ile, Pro, Trp, Ser, Tyr, Cys, Met, Asp, Gln, Glu, Thr, Arg and His or any combination thereof at each occurrence. In an embodiment, n is an integer of 1-50, preferably an integer of 1-30, preferably an integer of 1-20, preferably an integer of 1-10, and preferably 1, 2, 3, 4, 5, 6, 7, 8, 9 and 10. In an embodiment, -L²- is -Gly-Gly-Gly-.

In an embodiment, L¹ comprises a cleavable sequence containing an amino acid sequence that is enzymatically cleavable, wherein the amino acid sequence contains 1, 2, 3, 4, 5, 6, 7, 8, 9 or 10 amino acids. In an embodiment, the amino acid sequence that is enzymatically cleavable is selected from -Gly-Gly-Phe-Gly- (SEQ ID No. 6), -Phe-Lys-, -Val-Cit-, -Val-Lys-, -Gly-Phe-Leu-Gly-(SEQ ID No. 7), -Ala-Leu-Ala-Leu- (SEQ ID No. 8), -Ala-Ala-Ala- or a combination thereof; preferably, the amino acid sequence that is enzymatically cleavable is -Gly-Gly-Phe-Gly-. In an embodiment, -L¹- is -Gly-Gly-Phe-Gly-. In an embodiment, L¹ is any one of Val, Cit, Phe, Lys, Gly, Ala, Leu, Asn or any combination thereof, preferably, -Gly-Gly-Phe-Gly-, -Phe-Lys-, -Val-Cit-, -Val-Lys-, -Gly-Phe-Leu-Gly-, -Ala-Leu-Ala-Leu-, -Ala-Ala-Ala- or a combination thereof. In an embodiment, -L¹- is -Val-Cit-. In an embodiment, L¹ further contains a self-immolative spacer (Sp1).

In an embodiment, Sp1 is selected from PABC (p-aminobenzyloxycarbonyl), acetal, heteroacetal or a combination thereof; preferably, Sp1 is PABC. In an embodiment, -L¹- is -Val-Cit-PABC-.

In an embodiment, -L²-L¹- is —HN—(C₂H₄—O)ₘ—(CH₂)ₚ—(CO)-Gly-Gly-Phe-Gly-. In an embodiment, -L²-L¹- is —HN—(C₂H₄—O)ₘ—(CH₂)ₚ—(CO)-Val-Cit-PABC-.

In an embodiment, Ld2 and each Ld1 are independently selected from a bond or

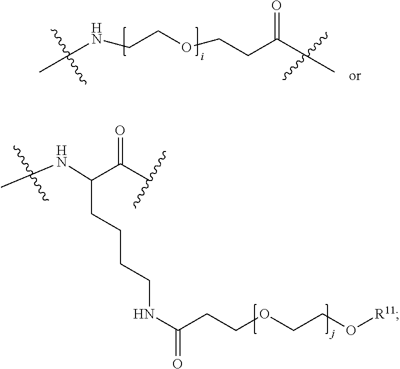

wherein i and j are independently selected from integers from 1 to 100.

In an embodiment, Ld1 is

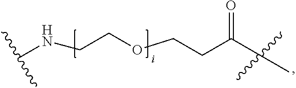

wherein i is an integer of 1 to 100. In an embodiment, Ld2 is

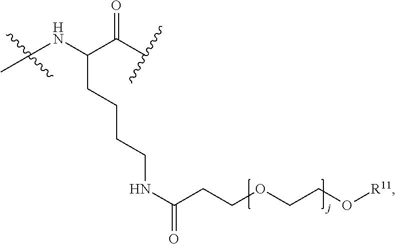

wherein j is an integer of 1 to 100.

In an embodiment, each i and j are independently an integer of 1 to 20. In an embodiment, each i and j are independently an integer of 1 to 12.

In an embodiment, each i is independently an integer of 2 to 8; particularly 4.

In an embodiment, each j is independently an integer of 8 to 12; particularly 12.

In an embodiment, Ld2 and each Ld1 are independently a bond; or a PEG fragment of a certain length (denoted as -(PEG)ᵢ-) with an amino and a carbonyl at the two ends respectively, or one or more natural amino acids independently unsubstituted or substituted with a PEG fragment of a certain length (denoted as —CO—(PEG)ⱼ-) on the side chain.

In an embodiment, -(PEG)ᵢ- comprises —(O—C₂H₄)ᵢ— or —(C₂H₄—O)ᵢ—, and an optional additional C₁₋₁₀ alkylene at one end; -(PEG)ⱼ- comprises —(O—C₂H₄)ⱼ— or —(C₂H₄—O)ⱼ—, and an optional additional C₁₋₁₀ alkylene at one end. In a specific embodiment, -(PEG)ᵢ- comprises —C₂H₄—(O—C₂H₄)ᵢ— or —(C₂H₄—O)ᵢ—C₂H₄—.

In an embodiment, a linker-payload compound, which has the structure of formula (I), is selected from the following structures:

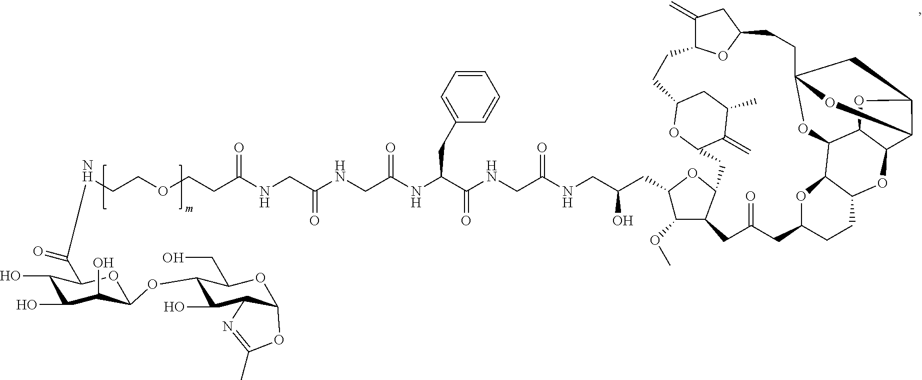

wherein,
m is an integer of 1, 2, 3, 4, 5, 6, 7, 8, 9 and 10; preferably an integer oft, 2, 3, 4 or 5;

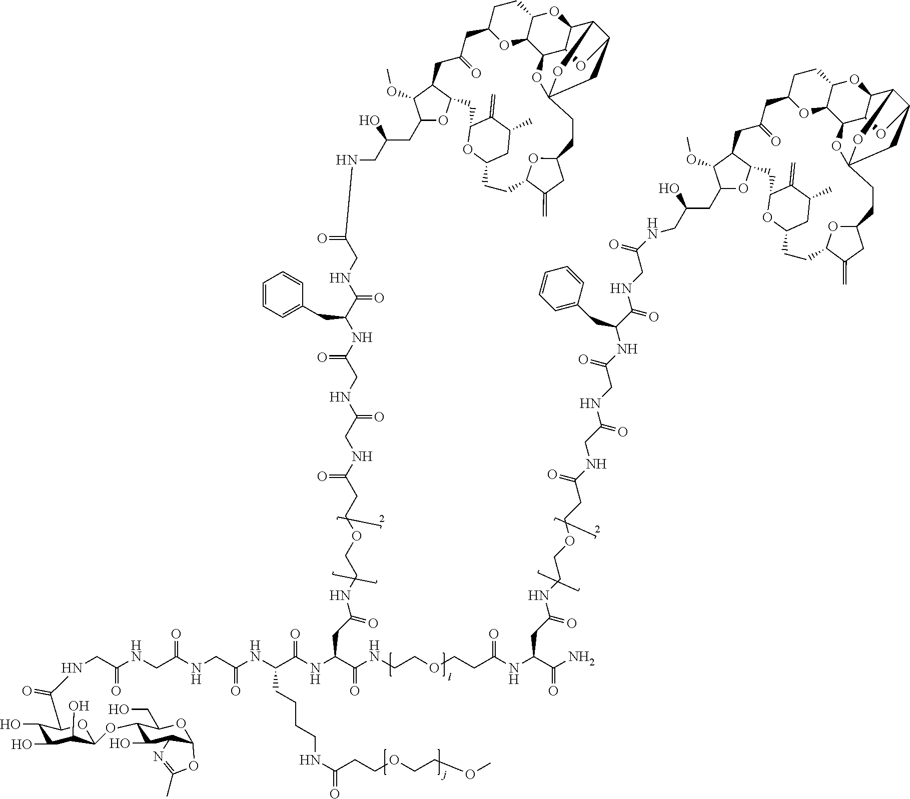

wherein,
each m is independently an integer of 1, 2, 3, 4, 5, 6, 7, 8, 9 and 10; preferably an integer of 1, 2, 3 or 4;
i is independently an integer from 1 to 100 (for example, 1, 2, 3, 4, 5, 6, 7, 8, 9, 10, 11 or 12);
10 j is independently an integer from 1 to 100 (for example, 1, 2, 3, 4, 5, 6, 7, 8, 9, 10, 11 or 12);

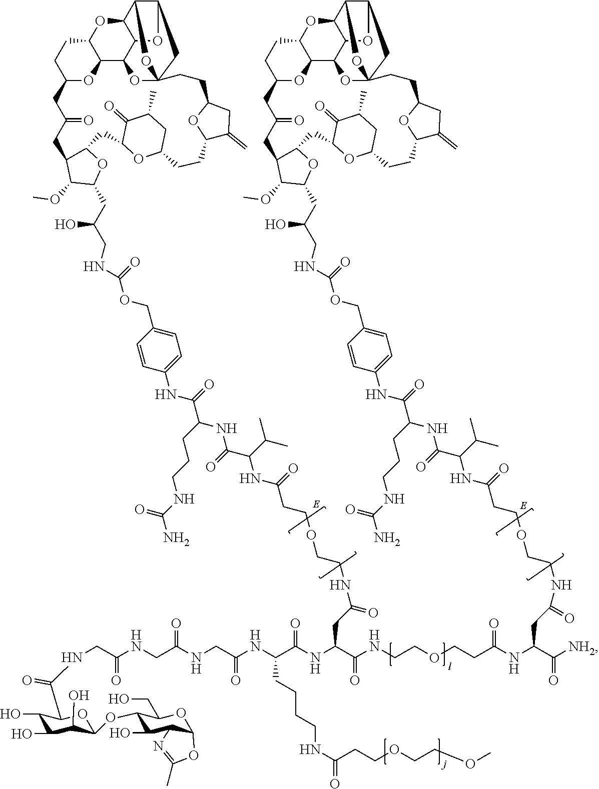

wherein,
each m is independently an integer of 1, 2, 3, 4, 5, 6, 7, 8, 9 and 10; preferably an integer of 1, 2, 3 or 4;
i is independently an integer from 1 to 100 (for example, 1, 2, 3, 4, 5, 6, 7, 8, 9, 10, 11 or 12);
j is independently an integer from 1 to 100 (for example, 1, 2, 3, 4, 5, 6, 7, 8, 9, 10, 11 or 12).

In another embodiment, a linker-payload compound, which has the structure of formula (I), is selected from the following structures:

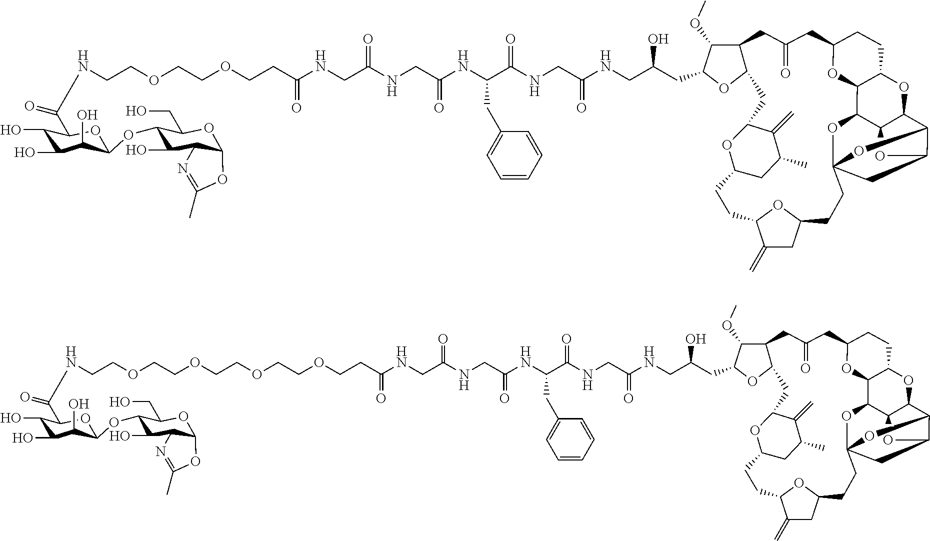

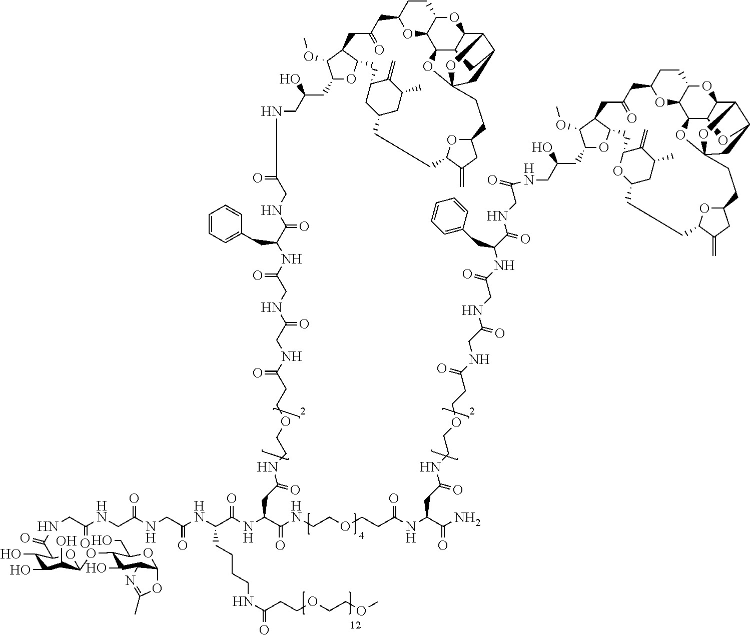

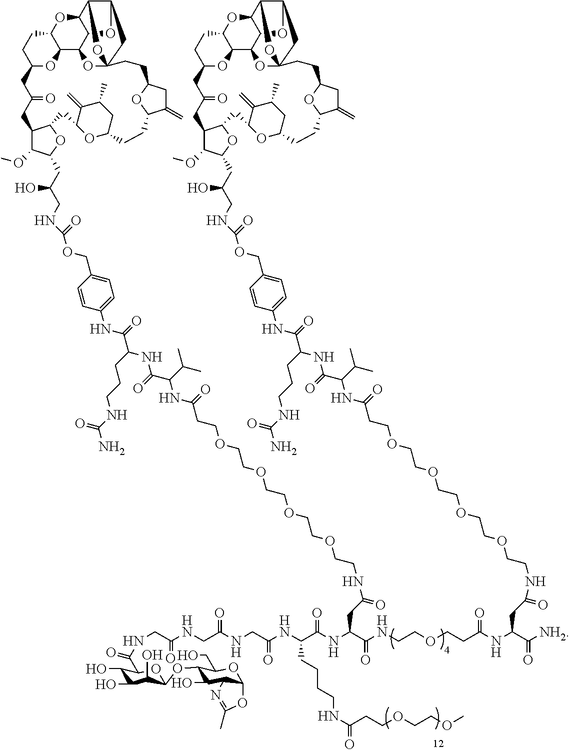

In another aspect, the disclosure provides a linker-payload compound, which is obtained by a method comprising:
(i) oxidizing the primary alcohol at the 6-position of the terminal hexose unit in disaccharide containing the terminal hexose unit and the terminal N-acetylglucosamine (GlcNAc) unit into a carboxyl group to obtain an intermediate compound (a) having a carboxyl group;
(ii) reacting carboxyl groups in the intermediate compound (a) obtained in step (i) with reactive groups in the linker-payload compound (b) with reactive groups at the end to obtain the linker-payload compound.

In an embodiment of another aspect, the monosaccharide moiety in the disaccharide is connected by a β-(1→4) glycoside bond.

In an embodiment of the another aspect, the disaccharide in step (i) has the following structure:

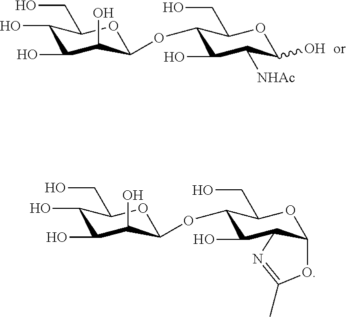

In an embodiment of the another aspect, the reactive group of the linker-payload compound (b) with a reactive group at the end in the step (ii) is amino.

In an embodiment of the another aspect, the method may further include the step of converting the intermediate compound (a) having a carboxyl group into an acyl halide, and further reacting with the linker-payload compound (b) with a reactive group at the end to obtain the linker-payload compound.

In an embodiment of the another aspect, the linker-payload compound is a linker-payload compound as defined in the first aspect or the respective embodiments thereof.

### Preparation Process of the Linker-Payload Compound

In a second aspect, the present disclosure provides a process for preparing a linker-payload compound having the structure of formula (I), wherein the variables are as defined in the first aspect or the respective embodiments thereof unless otherwise specified.

In an embodiment, the process includes the step of amide formation reaction between

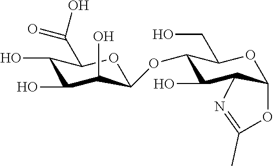

and L′-(P)ₜ to form

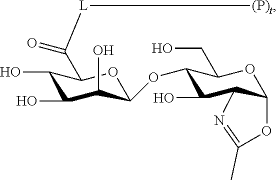

wherein L′ is as defined for L in the first aspect or in various embodiments thereof, except that —NH— in L connected to

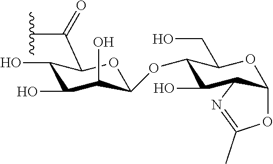

### is H₂N-in L′.

In an embodiment, when the structure of

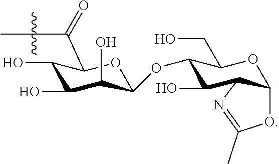

in the prepared linker-payload compound of formula (I) is

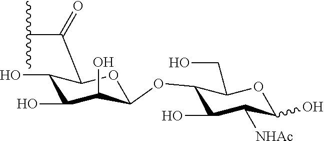

the method may further include the step of condensing

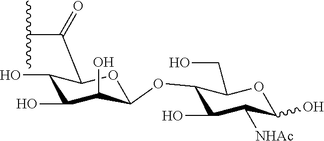

into

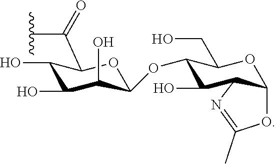

In an embodiment, the condensation is carried out in the presence of water, base and 2-chloro-1,3-dimethylimidazoline chloride (DMC, CAS: 37091-73-9). In an embodiment, the base is an inorganic base or an organic base. In an embodiment, the inorganic base is potassium carbonate, potassium phosphate, etc. In an embodiment, the inorganic base is potassium carbonate, potassium phosphate, etc. In an embodiment, the organic base is an amine, such as a tertiary amine, such as triethylamine (Et₃N).

In an embodiment, the amide formation reaction is carried out in the presence of an organic solvent, an organic base and a condensation reagent. In an embodiment, the organic solvent is selected from N,N-dimethylformamide (DMF) and N,N-dimethylacetamide (DMA). In an embodiment, the organic base is selected from diisopropylethylamine (DIPEA) and N-methylmorpholine (NMM). In an embodiment, the condensation reagent is selected from HATU, HBTU, TBTU and PyBOP.

In an embodiment,

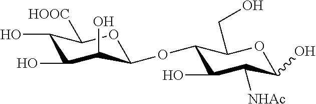

is prepared by

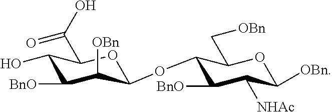

In an embodiment, the preparation is carried out under the condition of debenzylation protection of the substrate. In an embodiment, the deprotection is carried out in the presence of hydrogen and palladium catalyst. In an embodiment, the palladium catalyst is selected from palladium carbon and palladium carbon hydroxide.

In an embodiment,

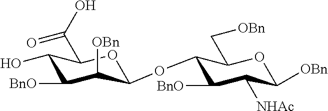

is prepared by

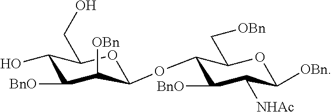

In an embodiment, the preparation is carried out in the presence of an oxidizing agent and an optional oxidation catalyst. In an embodiment, the oxidant is iodobenzene diacetate. In an embodiment, the oxidation catalyst is 2,2,6,6-tetramethyl-1-piperidine oxide.

In an embodiment,

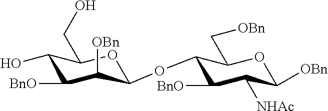

is prepared by

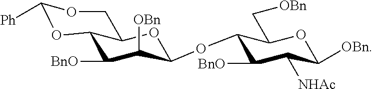

In an embodiment, the preparation is carried out in the presence of an acid. In an embodiment, the acid is p-toluenesulfonic acid.

In an embodiment,

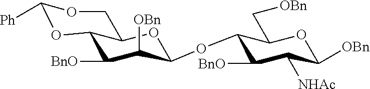

is prepared by

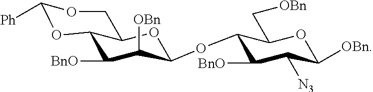

In an embodiment, the preparation is carried out in the presence of thioacetic acid (AcSH) and an organic solvent. In an embodiment, that organic solvent is selected from chloroform and pyridine or a mixture thereof.

In an embodiment,

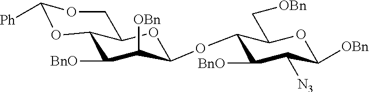

is prepared by

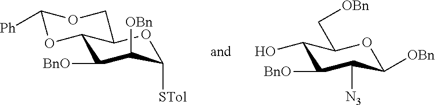

by forming a β-(1→4) glycosidic bond. In an embodiment, the preparation is carried out under glycosidic bond formation conditions. In an embodiment, the glycosidic bond formation conditions include the use of trifluoromethanesulfonic anhydride under anhydrous conditions.

### Antibody-Drug Conjugate

In a third aspect, the disclosure provides an antibody-drug conjugate, which has the structure of formula (II):

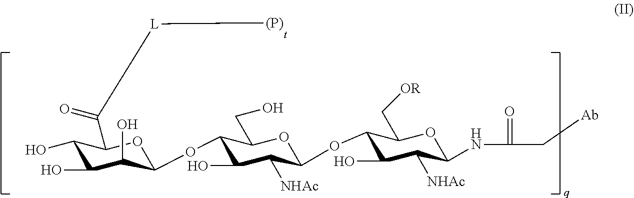

wherein
R is hydrogen or α-L-fucosyl;
q is 1 or 2;
Ab is an antibody or antigen-binding fragment;
L is a connecting moiety comprising —NH—, and L is directly connected with the carbonyl group in the disaccharide moiety via the —NH—, wherein when L is a linear connecting moiety, it is connected with one P moiety, and t is 1; or when L is a branched connecting moiety, each of the branches is connected with one P moiety at most, and t is an integer greater than 1 (for example, 2, 3, 4, 5, 6, 7, 8, 9 or 10);
P is a payload with the structure of

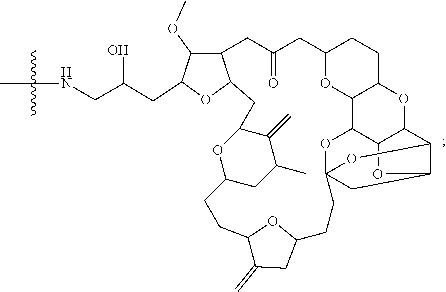 [SMILES: *NCC(O)CC1OC2CC3OC(CCC4OC(CCC56CC7OC8C(OC9CCC(CC(=O)CC2C1OC)OC9C8O5)C7O6)CC4=C)CC(C)C3=C]

preferably, P is Eribulin with the structure of

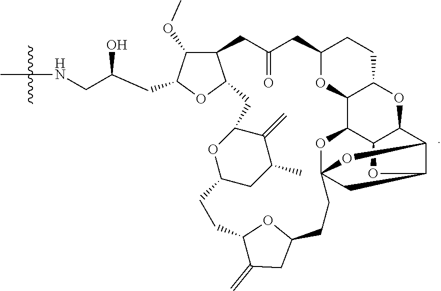 [SMILES: *NC[C@@H](O)C[C@H]1O[C@H]2C[C@H]3O[C@@H](CC[C@@H]4O[C@@H](CC[C@@]56C[C@H]7O[C@H]8[C@@H](O5)[C@H]5O[C@H](CC[C@@H]5O[C@H]8[C@H]7O6)CC(=O)C[C@@H]2[C@H]1OC)CC4=C)C[C@@H](C)C3=C]

In an embodiment, each the monosaccharide moiety is connected by a β-(1→4) glycoside bond.

In an embodiment, the —NHC(O)CH₂— in the formula (II) is derived from asparagine at position 297 in the Fc region of the antibody.

In an embodiment, the target identified by Ab is ErbB2/HER2.

In an embodiment, Ab is a monoclonal antibody. In an embodiment, Ab is an anti-human HER2 antibody or an antigen-binding fragment thereof. Examples of anti-human HER2 antibodies include but are not limited to Pertuzumab and Trastuzumab. In an embodiment, Ab is Trastuzumab.

In an embodiment, an antibody-drug conjugate based on the site-specific conjugating of the N-glycosylation site in Fc region of the antibody has the structure of formula (II):

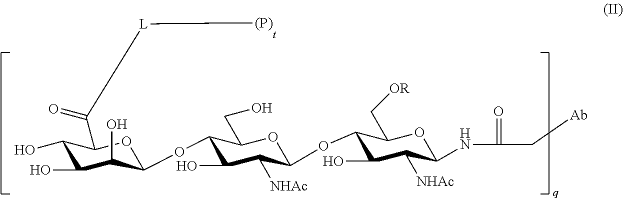

wherein
R is hydrogen or α-L-fucosyl;
q is 1 or 2, for example, q is 2;
Ab is an antibody or antigen-binding fragment; preferably an anti-ErbB2/HER2 antibody (for example, Trastuzumab);
(P)ₜ-L- is selected from:

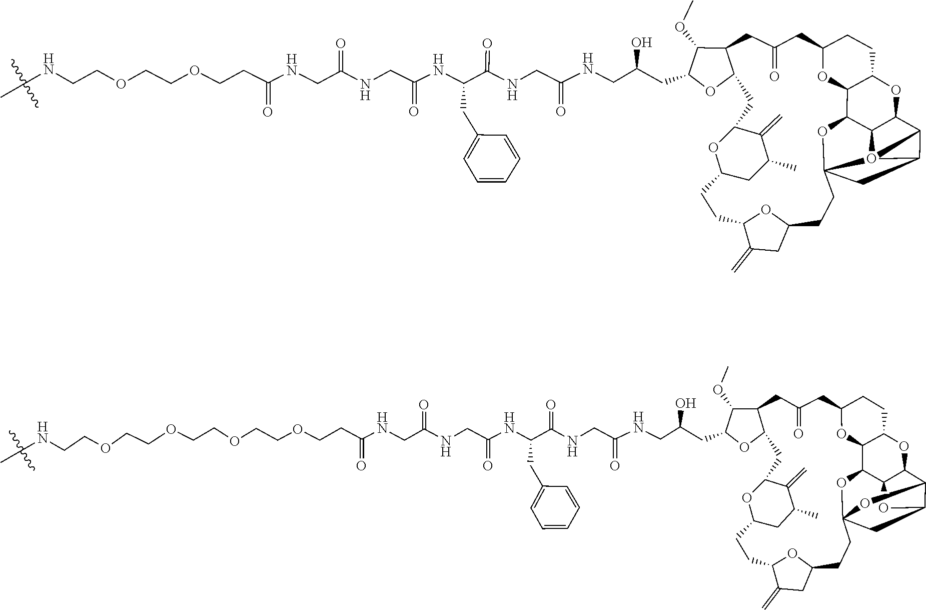

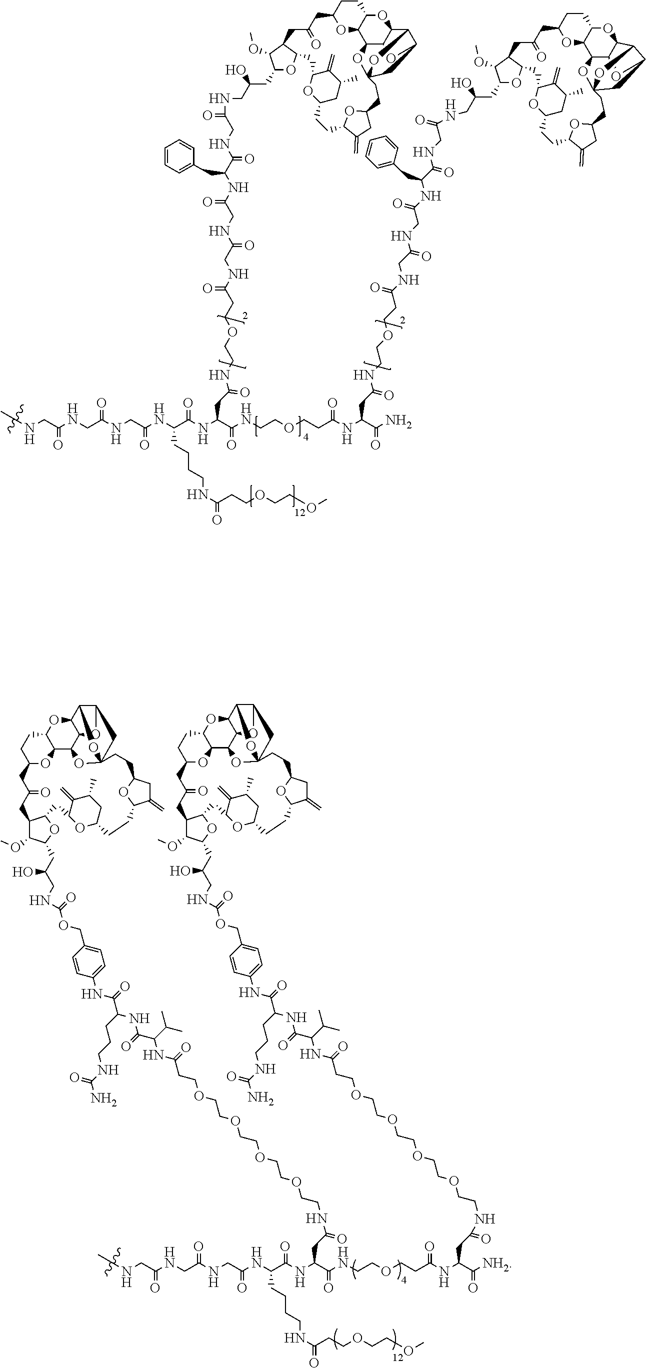

In an embodiment, R in the formula (II) is α-L-fucosyl;
q is 2;
Ab is Trastuzumab;
(P)ₜ-L- is

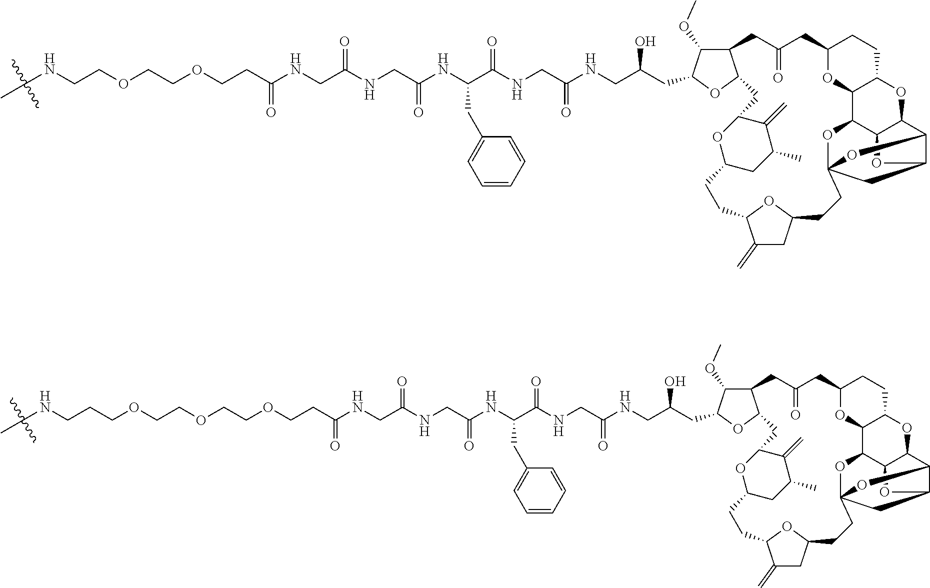 [SMILES: *NCCOCCOCCC(=O)NCC(=O)NCC(=O)N[C@@H](Cc1ccccc1)C(=O)NCC(=O)NC[C@@H](O)C[C@H]1O[C@H]2C[C@H]3O[C@@H](CC[C@@H]4O[C@@H](CC[C@@]56C[C@H]7O[C@H]8[C@@H](O5)[C@H]5O[C@H](CC[C@@H]5O[C@H]8[C@H]7O6)CC(=O)C[C@@H]2[C@H]1OC)CC4=C)C[C@@H](C)C3=C ; *NCCCOCCOCCOCCC(=O)NCC(=O)NCC(=O)N[C@@H](Cc1ccccc1)C(=O)NCC(=O)NC[C@@H](O)C[C@H]1O[C@H]2C[C@H]3O[C@@H](CC[C@@H]4O[C@@H](CC[C@@]56C[C@H]7O[C@H]8[C@@H](O5)[C@H]5O[C@H](CC[C@@H]5O[C@H]8[C@H]7O6)CC(=O)C[C@@H]2[C@H]1OC)CC4=C)C[C@@H](C)C3=C]

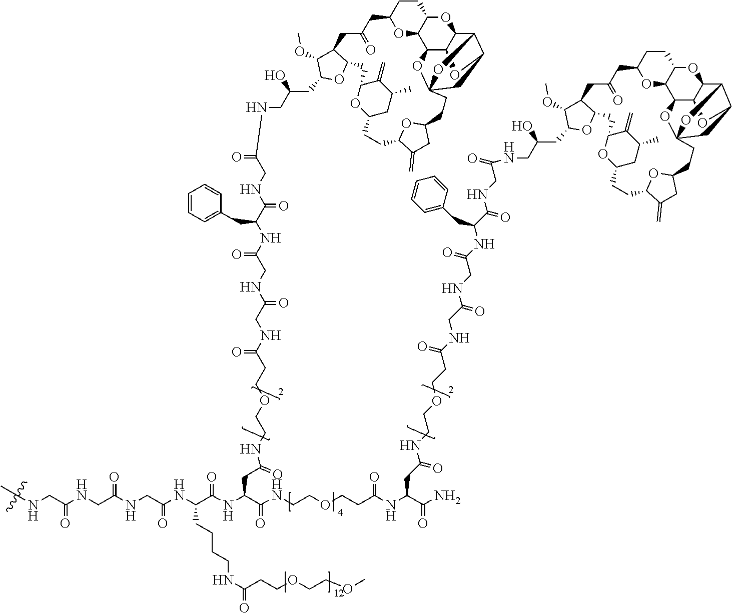

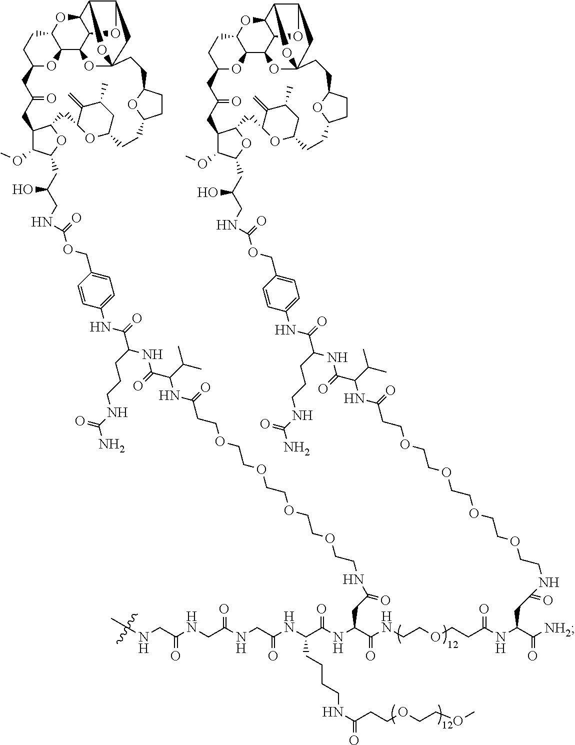

the —NHC(O)CH₂— in the formula (II) is derived from asparagine at position 297 in the Fc region of the antibody.

### Preparation Method of Antibody-Drug Conjugate

In a fourth aspect, the present disclosure provides a method for preparing an antibody-drug conjugate having the formula (II), which comprises conjugating a linker-payload compound having the formula (I) with an antibody Ab, wherein the variables are as defined in the third aspect or various embodiments thereof unless otherwise specified.

In an embodiment, the method comprises:
a) cleaving off the N-glycan chain part of the antibody Ab to afford a remodeled antibody of which the Fe region N-glycosylation site is N-acetylglucosamine or fucosyl-aX-1,6-N-acetylglucosamine, under the catalysis of glycosidase or a mutant thereof;
b) conjugating the remodeled antibody with the linker-payload compound described above under the catalysis of glycosidase or a mutant thereof;
wherein, the glycosidase or the mutant thereof used in step a) and step b) is identical or different.

In an embodiment, the glycosidase or a mutant thereof used in steps a) and b) is fucose hydrolase, N-acetylglucosamine endohydrolase or a mutant thereof. In an embodiment, the N-acetylglucosamine endohydrolase comprises at least one of Endo-S (Streptococcus pyogenes endoglycosidase —S), Endo —F3 (Elizabeth Kingia miricola endoglycosidase-F3), Endo-S2(Endoglycosidase-S2, Streptococcus pyogenes endoglycosidase —S2), Endo-Sd (Endoglycosidase-Sd, Streptococcus pyogenes endoglycosidase-Sd) and Endo-CC (Endoglycosidase-CC, Streptococcus pyogenes endoglycosidase-CC); preferably, endoglycosidase is Endo H, Endo D, Endo F2, Endo F3, Endo M, Endo CCI, Endo CC2, Endo Om, Endo S and Endo S2. In an embodiment, the enzyme is Endo S2.

In an embodiment, steps a) and b) are carried out by one-pot enzymatic catalysis.

In an embodiment, steps a) and b) are carried out by one-pot enzymatic catalysis, and the enzyme is Endo S2.

### Pharmaceutical Composition and Pharmaceutical Formulation

Another object of the disclosure is to provide a pharmaceutical composition comprising a prophylactically or therapeutically effective amount of a conjugate of the present disclosure, and at least one pharmaceutically acceptable carrier.

The pharmaceutical composition of the present disclosure may be administered in any manner as long as it achieves the effect of preventing, alleviating, preventing or curing the symptoms of a human or animal. For example, various suitable dosage forms can be prepared according to the administration route, especially injections such as lyophilized powder for injection, injection, or sterile powder for injection.

The term “pharmaceutically acceptable” means that when contacted with tissues of the patient within the scope of normal medical judgment, no undue toxicity, irritation or allergic reaction, etc. shall arise, having reasonable advantage-disadvantage ratios and effective for the intended use.

The term pharmaceutically acceptable carrier refers to those carrier materials which are pharmaceutically acceptable and which do not interfere with the bioactivities and properties of the conjugate. Examples of aqueous carriers include but are not limited to buffered saline, and the like. The pharmaceutically acceptable carrier also includes carrier materials which brings the composition close to physiological conditions, such as pH adjusting agents, buffering agents, toxicity adjusting agents and the like, and sodium acetate, sodium chloride, potassium chloride, calcium chloride, sodium lactate, and the like.

In an embodiment, the pharmaceutical composition of the present disclosure has a drug to antibody ratio (DAR) of an integer or non-integer of about 1 to about 20, such as about 1 to about 10, about 1 to about 8, about 1 to about 6, about 1 to about 4, about 1 to about 3, about 1 to about 2.5, about 1 to about 2. In a particular embodiment, the conjugate of the present disclosure has a DAR of about 2, about 4, about 6 or about 8. In a particular embodiment, the conjugate of the present disclosure has a DAR of about 1.76, about 1.81, about 3.82 or about 3.86.

### Treatment Method and Use

The antibody-drug conjugates of the present disclosure are useful for the treatment of a tumor and/or an autoimmune disease. Tumors susceptible to antibody-drug conjugate treatment include those characterized by specific tumor-associated antigens or cell surface receptors, and those will be recognized by the targeting molecule in the antibody-drug conjugate and can be killed by the payload/cytotoxin in the antibody-drug conjugate.

Accordingly, in yet another aspect, also provided is use of an antibody-drug conjugate of the present disclosure or a pharmaceutical composition of the present disclosure in the manufacture of a medicament for treating a disease, disorder, or condition selected from a tumor or an autoimmune disease.

In another aspect, provided is an antibody-drug conjugate of the present disclosure or a pharmaceutical composition of the present disclosure for use in the treatment of a tumor or an autoimmune disease.

In a further aspect, provided is a method of treating a tumor or an autoimmune disease, the method comprising administering to an individual in need thereof an effective amount of a antibody-drug conjugate of the present disclosure or a pharmaceutical composition of the present disclosure.

In an embodiment, the antibody-drug conjugate of the present disclosure formed by conjugation of the anti-human HER2 antibody and the small molecule cytotoxin can specifically bind to HER2 on the surface of the tumor cell and selectively kill the HER2-expressing tumor cells. In another embodiment, provided is use of a antibody-drug conjugate of the present disclosure or a pharmaceutical composition of the present disclosure in the manufacture of a medicament for treating a disease, disorder or condition selected from HER2-positive tumors. In a more preferable embodiment, the disease, disorder or condition is selected from the group consisting of breast cancer, gastric cancer, lung cancer, ovarian cancer, urothelial cancer, and the like.

The dosage of the antibody-drug conjugate administered to the subject can be adjusted to a considerable extent. The dosage can vary according to the particular route of administration and the needs of the subject, and can be determined by healthcare professionals.

### Beneficial Effects

The disclosure expands the range of ADC obtained by glycan chain remodeling technology because of the novel oligosaccharide structure forming amide bond. Under the expected circumstances, compounds with NH₂ (for example, compounds with L′-(P)ₜ structure, where P and t are as defined herein, and L′ is as defined for L herein, except that —NH— in L connected to

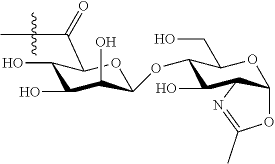

is H₂N-in L′) can form a linker-payload

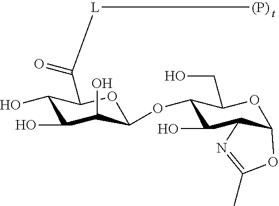

containing oligosaccharide linker through the disaccharide structure of present disclosure through a simple amide reaction. The ADC can be further formed with antibodies, and the conjugation of

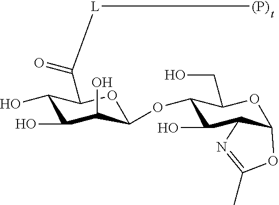

with antibodies can be carried out through one-pot enzymatic catalysis. In addition, the ADC with novel oligosaccharide structure forming amide bond provided by the present disclosure can be efficiently delivered to target cells and the payload can be efficiently released in the target cells. In the disclosure, a unique disaccharide carboxylic acid substrate (for example,

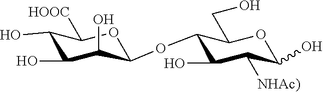

can be well connected with an amino compound in one step through a mild and compatible amide bonding mode, thus simplifying and accelerating the synthesis of linker-payload compound without additional reactions that may lead to many adverse by-products.

Because the linker-payload is formed by a simple amide reaction between a uniquely designed disaccharide substrate and a fragment containing Eribulin, the variety of the existing ADCs containing Eribulin is enriched. The novel linker-payload of the present disclosure can be applied to the novel site-specific conjugating of the antibody glycan chain catalyzed by enzymes technology platform, and realize one-step, site-specific, uniform and stable conjugation of Eribulin to antibody. The conjugation production process is simple and a large-scale production can be realized. Compared to the ADCs of Eribulin in the prior art, the uniformity, stability and safety of the ADCs prepared by the linker-payload of the present disclosure are greatly improved, especially the ADCs has excellent maximum tolerated dose (MTD), thereby the potential therapeutic window of Eribulin molecules in the ADCs is greatly broadened.

### EXAMPLES

Unless otherwise stated, the instruments and reagents used in the examples are commercially available. The reagents can be used directly without further purification.

### Example 1: Synthesis of Disaccharide Compound 1

### Step 1.1 Synthesis of Disaccharide Compound 1c

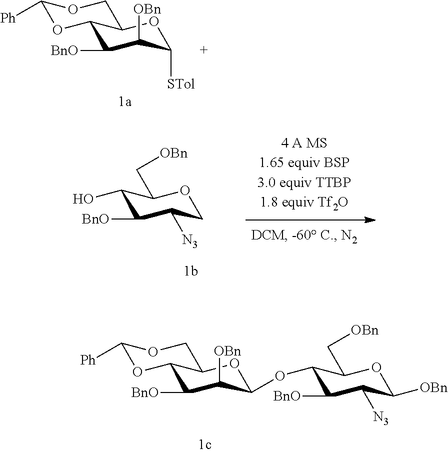

To an oven-dried 2-necked flask (100 mL) equipped with a Teflon-coated magnetic stir bar was added freshly activated 4 Å MS, capped with a sleeve stopper, the system was then baked with a heat gun under vacuum, cooled to ambient temperature, and then backfilled with nitrogen. Then Compound 1a (4.41 g, 7.96 mmol), anhydrous dichloromethane (30 mL) were added and stirred for 0.5 hour at room temperature (rt). Following the same protocol, Compound 1b (1.89 g, 3.98 mmol) was stirred and pre-dried in 20 mL anhydrous dichloromethane in the presence of activated molecular sieves. To the resulting reaction mixture of Compound 1a were added 1-(phenylthionyl) piperidine (BSP, 1.37 g, 6.56 mmol) and 2,4,6-tri-tert-butylpyrimidine (TTBP, 2.94 g, 11.94 mmol) at rt, and stirred for 20 minutes at the same temperature. Then the reaction flask was cooled to −65° C., followed by the dropwise addition of trifluoromethanesulfonic anhydride (1.2 mL, 7.16 mmol). After 2 minutes, the dichloromethane solution of the previously pre-dried Compound 1b was added to the system, and the resulting reaction mixture was stirred at −65° C. until the reaction was completed monitored by TLC (eluent: EtOAc/PE=1/8). Then the reaction mixture was quenched with saturated sodium bicarbonate solution (20 mL), extracted with dichloromethane (150 mL×3), the combined organic layer was washed with water and saturated salt water sequentially, dried over anhydrous sodium sulfate, filtered, concentrated under reduced pressure, and purified by flash column chromatography on silica gel (eluent: EtOAc/PE=1/12-1/10) to obtain Compound 1c as a colorless viscous oily liquid (2.72 g, 75.4% yield). ¹H NMR (400 MHz, chloroform-d) δ 7.55 (dd, J=7.6, 2.1 Hz, 2H), 7.51-7.28 (m, 28H), 5.60 (s, 1H), 5.14 (d, J=10.4 Hz, 1H), 5.00 (d, J=12.0 Hz, 1H), 4.94 (d, J=11.8 Hz, 1H), 4.90-4.80 (m, 2H), 4.76 (d, J=12.0 Hz, 1H), 4.74-4.63 (m, 3H), 4.57 (s, 1H), 4.48 (d, J=12.1 Hz, 1H), 4.37 (d, J=8.1 Hz, 1H), 4.22-4.10 (m, 2H), 4.05 (t, J=9.3 Hz, 1H), 3.80 (d, J=3.1 Hz, 1H), 3.72 (dd, J=11.2, 2.2 Hz, 1H), 3.67-3.52 (m, 3H), 3.49 (dd, J=9.8, 3.1 Hz, 1H), 3.41 (t, J=9.3 Hz, 1H), 3.35 (dt, J=9.8, 2.9 Hz, 1H), 3.16 (td, J=9.7, 4.8 Hz, 1H). MS (ESI) calcd for C₅₄H₅₆N₃O₁₀⁺[M+H]⁺: 906.4. Found: 906.7.

### Step 1.2 Synthesis of Compound 1d

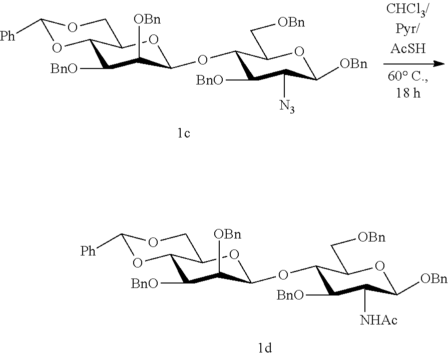

To a 25 mL Schlenk reaction flask were added Compound Ic (228 mg, 0.252 mmol), chloroform (1.2 mL), pyridine (1 mL), and AcSH (1.2 mL) successively. The tube was sealed and the system was stirred at 60° C. for 18 hours. Then the reaction system was cooled to rt, concentrated under reduced pressure to remove most of the solvents, and then water (30 mL) was added, the resulting mixture was quenched with ethyl acetate (20 mL×3), the combined organic layer was washed sequentially with saturated sodium bicarbonate solution (30 mL), 1 M hydrochloric acid (10 mL×4) and saturated sodium bicarbonate solution (30 mL). The resulting organic phase was dried over anhydrous sodium sulfate, concentrated and purified by flash column chromatography on silica gel (eluent: EtOAc/PE=1/10-1/1/) to offord Compound 1d as a white solid (200 mg, 86% yield). ¹H NMR (400 MHz, chloroform-d) δ 7.57-7.49 (m, 2H), 7.48-7.39 (m, 5H), 7.39-7.24 (m, 23H), 5.80 (d, J=8.1 Hz, 1H), 5.59 (s, 1H), 5.00 (d, J=6.7 Hz, 1H), 4.97-4.87 (m, 3H), 4.87-4.75 (m, 2H), 4.69-4.59 (m, 4H), 4.57 (s, 1H), 4.45 (d, J=12.0 Hz, 1H), 4.19-4.05 (m, 3H), 3.96 (t, J=7.3 Hz, 1H), 3.85-3.76 (m, 2H), 3.73-3.59 (m, 4H), 3.49 (dd, J=9.8, 3.1 Hz, 1H), 3.18 (td, J=9.7, 4.8 Hz, 1H), 1.77 (d, J=1.2 Hz, 3H). MS (ESI) calcd for C₅₆H₆₀NO₁₁⁺ [M+H]⁺: 922.4. Found: 922.4.

### Step 1.3 Synthesis of Compound 1e

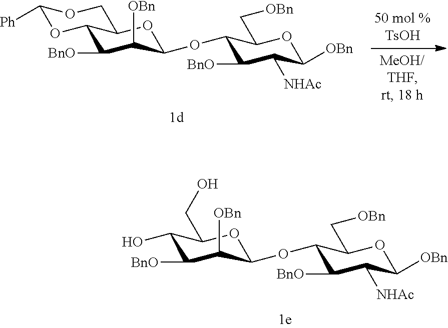

To a solution of Compound id (168 mg, 0.168 mmol) in methanol/tetrahydrofuran (1:1, 10 mL) was added p-toluenesulfonic acid monohydrate (35 mg, 0.168 mmol), the resulting mixture was stirred overnight at rt. After completion of the reaction, the mixture was quenched with saturated sodium bicarbonate solution (20 mL), extracted with dichloromethane (20 mL×3), the combined organic layer was dried over anhydrous sodium sulfate, filtered, concentrated under reduced pressure, and purified by flash column chromatography on silica gel (eluent: MeOH/DCM=1/20) to give Compound 1e as a white solid (136 mg, yield 89%). ¹H NMR (400 MHz, chloroform-d) δ 7.43-7.24 (m, 29H), 5.76 (d, J=7.9 Hz, 1H), 5.01 (d, J=6.9 Hz, 1H), 4.94 (dd, J=11.8, 2.3 Hz, 2H), 4.85 (d, J=11.7 Hz, 1H), 4.77 (d, J=11.8 Hz, 1H), 4.70-4.59 (m, 3H), 4.58-4.47 (m, 3H), 4.36 (d, J=11.7 Hz, 1H), 4.20 (t, J=7.9 Hz, 1H), 3.94 (t, J=7.5 Hz, 1H), 3.91-3.80 (m, 3H), 3.80-3.71 (m, 3H), 3.63-3.50 (m, 2H), 3.22-3.14 (m, 2H), 1.78 (s, 3H). MS (ESI) calcd for C₄₉H₅₆NO₁₁⁺ [M+H]⁺: 834.4. Found 834.5.

### Step 1.4 Synthesis of Compound 1f

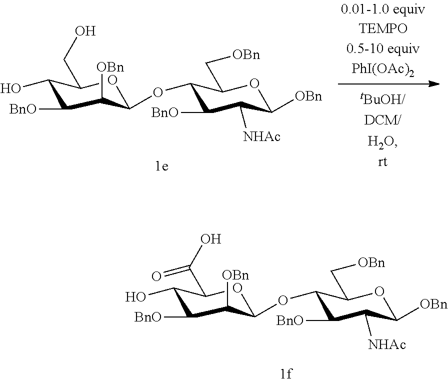

To a round flask (100 mL) were added Compound 1e (130 mg, 0.156 mmol), dichloromethane, tert-butanol, water, iodobenzene diacetate (0.5-10 eq.) and 2,2,6,6-tetramethyl-1-piperidine oxide (0.01-1 eq.), the resulting reaction mixture was stirred overnight at rt. Upon completion, the reaction mixture was extracted with dichloromethane, and the combined organic phases were dried with anhydrous sodium sulfate, filtered, concentrated and purified by flash column chromatography on silical gel to give Compound 1f as a white solid (100 mg, 75.1% yield). ¹H NMR (400 MHz, chloroform-d) δ 7.42-7.31 (m, 18H), 7.30-7.27 (m, 2H), 7.25-7.20 (m, 3H), 7.15 (dd, J=6.7, 2.9 Hz, 2H), 6.06 (d, J=8.3 Hz, 1H), 4.96 (d, J=6.6 Hz, 1H), 4.93 (d, J=11.9 Hz, 1H), 4.86 (d, J=11.1 Hz, 1H), 4.81 (d, J=12.1 Hz, 1H), 4.73 (d, J=11.1 Hz, 1H), 4.71-4.65 (m, 3H), 4.65-4.57 (m, 3H), 4.49 (s, 1H), 4.46 (d, J=12.0 Hz, 1H), 4.23 (t, J=7.1 Hz, 1H), 4.15 (t, J=9.5 Hz, 1H), 3.89 (t, J=6.5 Hz, 1H), 3.86-3.65 (m, 6H), 3.54 (d, J=9.7 Hz, 1H), 3.30 (dd, J=9.3, 2.8 Hz, 1H), 1.65 (s, 3H). MS (ESI) calcd for C₄₉H₅₂NO₁₂⁻ [M−H⁺]⁻: 846.3. Found: 846.3.

### Step 1.5 Synthesis of Compound 1

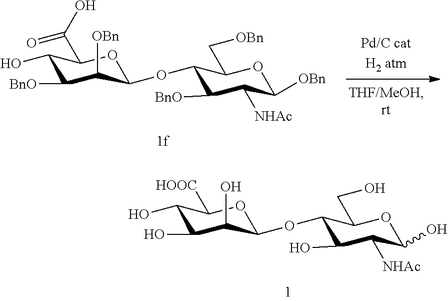

To a solution of Compound 1f (160 mg, 0.189 mmol) in tetrahydrofuran/methanol (1:1, 10 mL) was added palladium on carbon (10 wt. % loading), H₂ was bubbled through the solution for 5 mins, then the mixture was stirred under hydrogen atmosphere. Upon completion, the reaction mixture was filtered through Celite and washed with Methanol, the filtrate was then concentrated under reduced pressure, and dried in high vacuum to afford Compound 1 as a white solid (75 mg, 100% yield). MS (ESI) calcd for C₁₄H₂₂NO₁₂⁻ [M−H⁺]⁻: 396.1. Found: 396.1.

### Example 2: Synthesis of Compound 2

### Step 2.1 Synthesis of Compound 2a

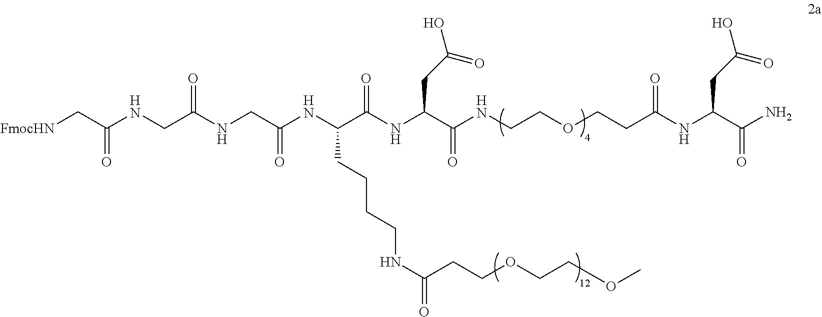

### Step 2.1.1 Synthesis of NH₂-Asp(OtBu)-Rink amide resin

400 g of Rink amide resin was weighed and fully swelled by 2400 mL of DCM. 2400 mL of deprotection reagent was added to remove Fmoc completely and then washed several times with DMF and DCM in room temperature. In the subsequent ninhydrin test, the resin was blue.

88.87 g Fmoc-Asp(OtBu)—OH and 29.19 g HOBT were weighed and dissolved in 2000 mL DMF and 80 mL DIC solution. After being placed in an ice bath at −10° C. for 0.5 h, it was slowly added into the reaction kettle with resin, and the reaction was stirred at room temperature for 2-5 h with nitrogen, and then filtered. The resin was washed with DMF and DCM successively and showed colorless or light yellow in the subsequent ninhydrin test.

2400 mL of deprotection reagent was added to remove Fmoc completely and then washed several times with DMF and DCM in room temperature. In the subsequent ninhydrin test, the resin showed blue color.

### Step 2.1.2 Synthesis of NH₂-PEG4-Asp(OtBu)-Rink Amide Resin

131.64 g Fmoc-PEG4-OH and 48.64 g HOBT were weighed and dissolved in 2000 mL DMF and 80.0 mL DIC solution. After being placed in an ice bath at −10° C. for 0.5 h, it was slowly added to the reaction kettle with resin, and the reaction was stirred at room temperature for 2-4 h with nitrogen, and then filtered. The resin was washed with DMF and DCM successively and showed colorless or light yellow in the subsequent ninhydrin test. 2400 mL of deprotection reagent was added to remove Fmoc completely and then washed several times with DMF and DCM in room temperature. In the subsequent ninhydrin test, the resin showed blue color.

### Step 2.1.3 Synthesis of NH₂-Asp(OtBu)-PEG4-Asp(OtBu)-Rink Amide Resin

222.18 g Fmoc-Asp(OtBu)—OH and 72.96 g HOBT were weighed and dissolved in 2000 mL DMF and 80 mL DIC solution. After being placed in an ice bath at −10° C. for 0.5 h, it was slowly added to the reaction kettle with resin, and the reaction was stirred at room temperature for 2-4 h with nitrogen, and then filtered. The resin was washed with DMF and DCM successively and showed colorless or light yellow in the subsequent ninhydrin test. 2400 mL of deprotection reagent was added to remove Fmoc completely and then washed several times with DMF and DCM in room temperature. In the subsequent ninhydrin test, the resin showed blue color.

### Step 2.1.4 Synthesis of Dde-Lys(NH₂)-Asp(OtBu)-PEG4-Asp(OtBu)-Rink Amide Resin

191.75 g Dde-Lys(Fmoc)-OH and 48.64 g HOBT were weighed and dissolved in 2000 mL DMF and 80.0 mL DIC solution. After being placed in an ice bath at −10° C. for 0.5 h, it was slowly added to the reaction kettle with resin, and the reaction was stirred at room temperature for 2-4 h with nitrogen, and then filtered. The resin was washed with DMF and DCM successively and showed colorless or light yellow in the subsequent ninhydrin test.

2400 mL of deprotection reagent was added to remove Fmoc completely and then washed several times with DMF and DCM in room temperature. In the subsequent ninhydrin test, the resin showed blue color.

### Step 2.1.5 Synthesis of Dde-Lys(mPEG12)-Asp(OtBu)-PEG4-Asp(OtBu)-Rink Amide Resin

170.84 g m-PEG12-CH₂CH₂COOH and 48.64 g HOBT were weighed and dissolved in 2000 mL DMF and 80.0 mL DIC solution. After being placed in an ice bath at −10° C. for 0.5 h, it was slowly added to the reaction kettle with resin, and the reaction was stirred at room temperature for 2-4 h with nitrogen, and then filtered. The resin was washed with DMF and DCM successively and showed colorless or light yellow in the subsequent ninhydrin test.

### Step 2.1.6 Synthesis of NH₂-Lys(PEG12)-Asp(OtBu)-PEG4-Asp(OtBu)-Rink Amide Resin

2400 mL of de-Dde reagent was added, and the reaction was stirred at room temperature under nitrogen for 0.5 h, and then filtered. After repeating the operation 3 times, the resin was washed with DMF and DCM successively and showed blue color in the subsequent ninhydrin test.

### Step 2.1.7 Synthesis of Fmoc-Gly-Gly-Gly-Lys(PEG12)-Asp(OtBu)-PEG4-Asp(OtBu)-Rink amide resin

111.08 g Fmoc-Gly-Gly-Gly-OH and 48.64 g HOBT were weighed and dissolved in 2000 mL DMF and 80.0 mL DIC solution. After being placed in an ice bath at −10° C. for 0.5 h, it was slowly added to the reaction kettle with resin, and the reaction was stirred at room temperature for 2-4 h with nitrogen, and then filtered. The resin was washed with DMF and DCM successively and showed colorless or light yellow in the subsequent ninhydrin test. The resin peptide was washed three times with anhydrous ethanol, filtered and waited for cleavage.

### Step 2.1.8 Synthesis of Compound 2a

10000 mL cleavage reagent (TFA:TIS:H₂O=95:2.5:2.5) was added to the 10 L reactor and cooled to −10±2° C. The dried and weighed resin was added. The reaction was warmed to room temperature and stirred under nitrogen for 2-3 h. After that, the resin was filtered and washed once with 100 mL of TFA. The filtrate and washing solution were combined.

40 L of pre-cooled (below −10° C.) cold ether was added into product solution. The mixture was stirred for 10 minutes, and then the precipitate was centrifuged. The supernatant was discarded after centrifugation, and the precipitate was collected and washed with cold ether, and the precipitate was centrifuged again (Each time the centrifugation speed was set to 3600 rpm, the centrifugation time was 5 minutes, and the temperature of the centrifuge cavity was −5° C.).

The precipitate was purified by Prep-HPLC and lyophilized to afford Compound 2a.

### Step 2.2 Synthesis of Compound 2e

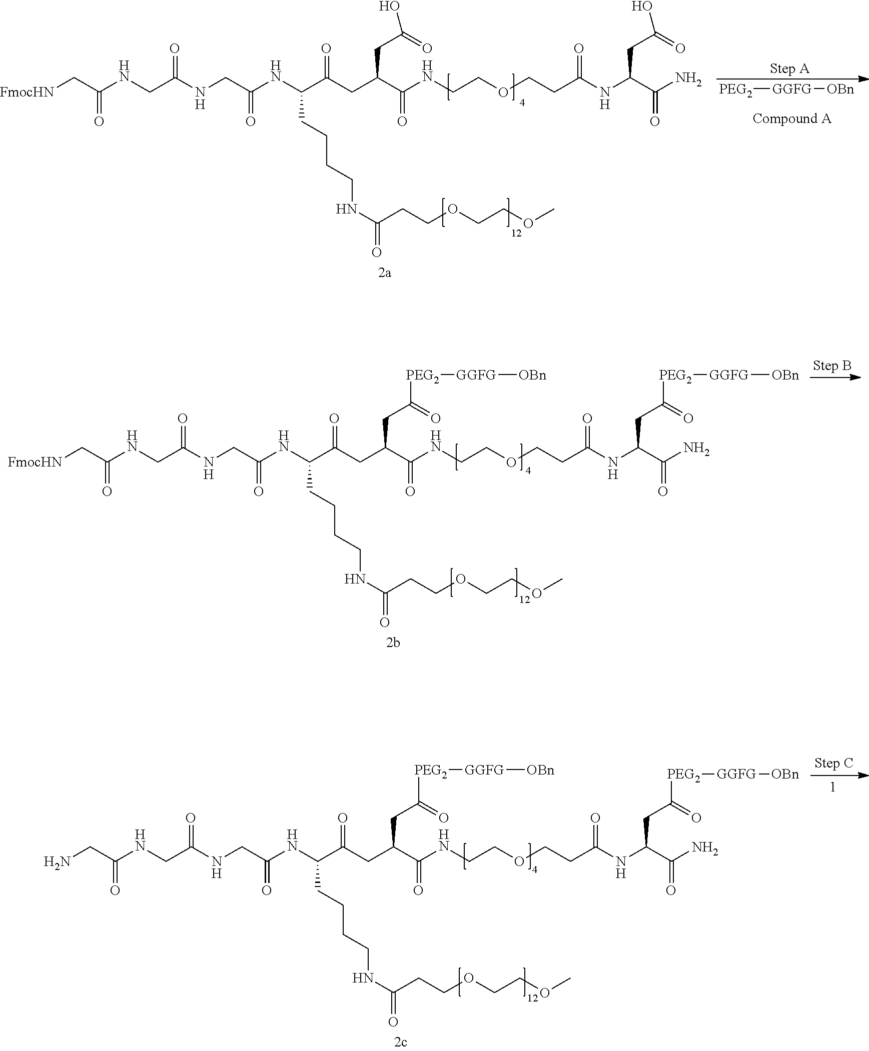

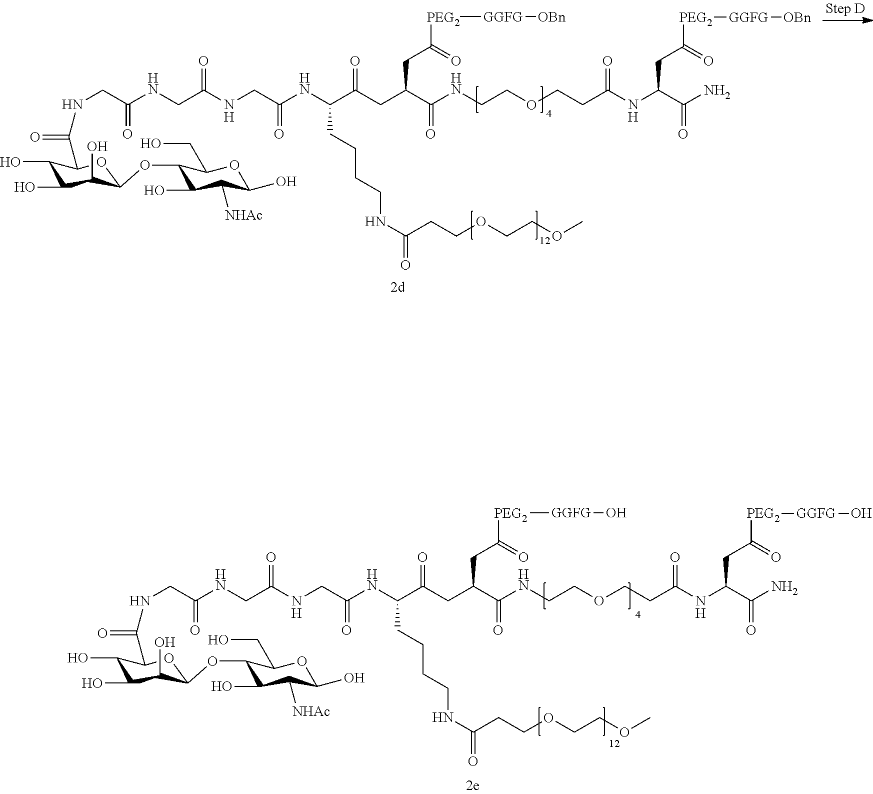

### Step 2.2.1 Synthesis of Compound 2b

To a stirred solution of Compound 2a and Compoud A (PEG2-GGFG-OBn) in DMF was added DIEA and HATU, the resulting reaction mixture was stirred at rt. Upon completion, the reaction mixture was directly used in the next step without any purification.

### Step 2.2.2 Synthesis of Compound 2c

To the reaction mixture of last step was added Et₂NH, the resulting mixture was allowed to stir for 15 min at rt, and then neutralized with 30% HOAc, directly purified by prep-HPLC to afford Compound 2c as a white solid (560 mg, 62% yield over 2 steps). MS (ESI) calcd for C₁₁₇H₁₈₃N₁₉O₄₃ [M+2H⁺]²⁺/2: 1272.1. Found: 1272.2.

### Step 2.2.3 Synthesis of Compound 2d

To a stirred solution of Compound 2c and Compound 1 in DMF was added DIEA and HATU, the resulting reaction mixture was stirred at rt, upon completion, the reaction mixture was purified by prep-HPLC to afford Compound 2d as a white solid (400 mg, 87% yield). MS calcd for C₁₃₁H₂₀₄N₂₀O₅₄ [(M+3H⁺+H₂O)]³⁺/3: 980.8. Found: 981.0.

### Step 2.2.4 Synthesis of Compound 2e

To a stirred solution of Compound 2d (400 mg) in water was added palladium hydroxide on carbon (10 wt. % loading). The air in the system was replaced with hydrogen for 3 times, the resulting reaction mixture was stirred at room temperature. Upon completion, the reaction solution was filtered and purified by prep-HPLC to afford Compound 2e as a white solid (240 mg, 64% yield). MS (ESI) calcd for C₁₁₇H₁₉₂N₂₀O₅₄ [M+2H⁺]²⁺/2: 1372.1. Found: 1372.29.

### Step 2.3 Synthesis of Compound 2

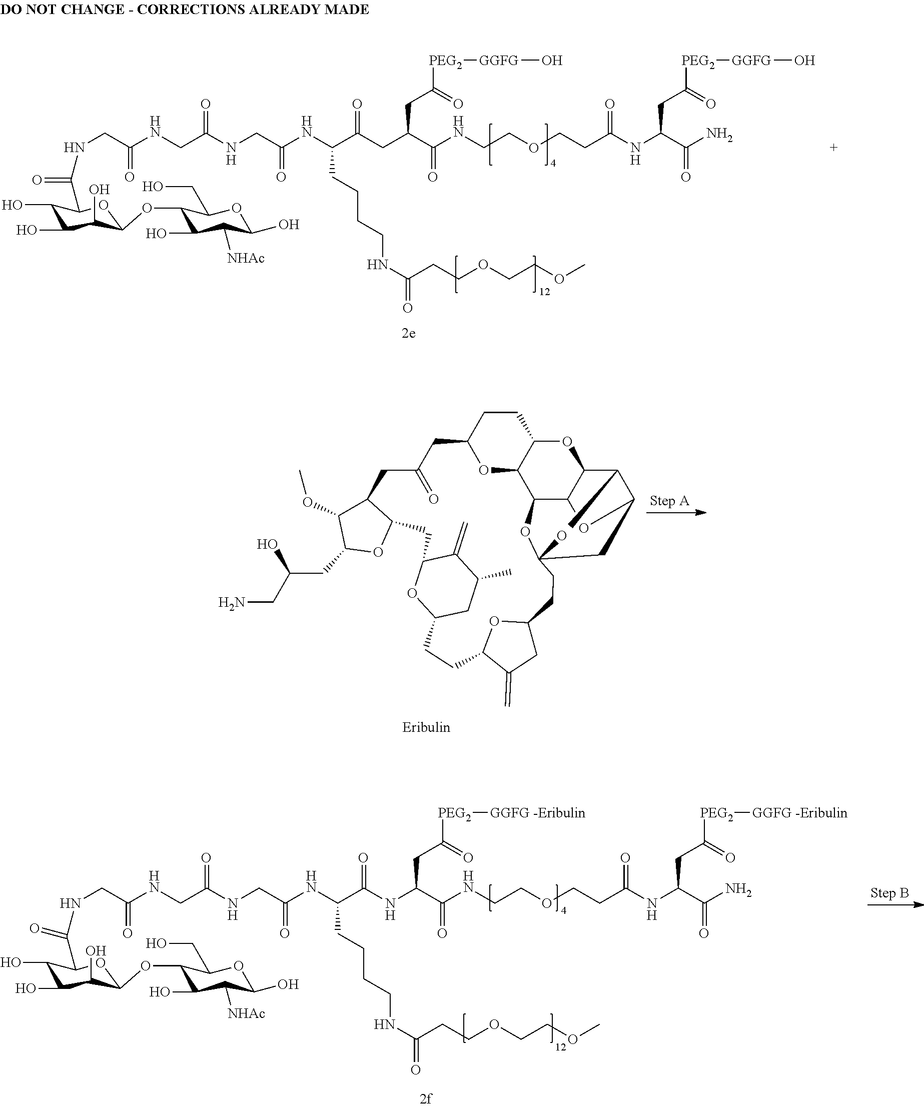

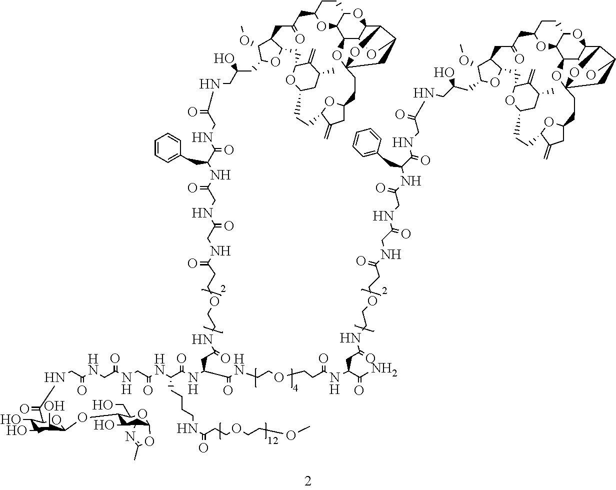

### Step 2.3.1 Synthesis of Compound 2f

To a stirred solution of Compound 2e and Eribulin mesylate in DMF were added DIEA and HATU, the resulting reaction mixture was stirred at rt. Upon completion, the reaction mixture was purified by prep-HPLC to yield Compound 2f as a white solid (245 mg, 67% yield) MS (ESI) calcd for C₁₉₇H₃₀₆N₂₂O₇₄ [M+3H⁺]³⁺/3: 1389.0. Found: 1389.1.

### Step 2.3.2: Synthesis of Compound 2

To a stirred solution of Compound 2f in water was added Et₃N and 2-Chloro-1,3-dimethylimidazolidinium chloride (DMC, CAS: 37091-73-9), the resulting mixture was stirred at certain temperature (e.g., −20° C. to 40° C.), after the completion of the reaction monitored by HPLC, the reaction mixture was directly purified by prep-HPLC to afford Compound 2 as a white solid (139 mg, 63% o yield). MS (ESI) calcd for C₁₉₇H₃₀₄N₂₂O₇₃ [M+3H⁺+H₂O]³⁺/3: 1389.0. Found: 1389.1.

### Example 3: Preparation of Compound 3

### Step 3.1 Synthesis of Compound 3a

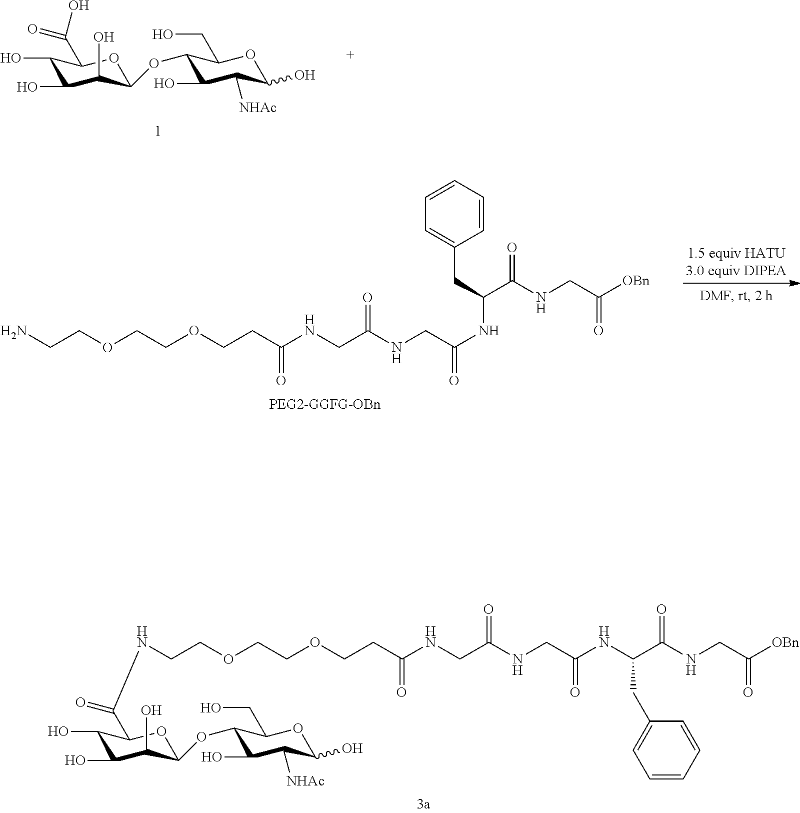

Compound 1 (51 mg, 0.128 mmol) and PEG2-GGFG-OBn (50 mg, 0.085 mmol) were weighed, added into an eggplant type flask, and dissolved in 1 mL of DMF at room temperature. HATU (48 mg, 0.128 mmol) and DIPEA (33 mg, 0.255 mmol) were sequentially added and the reaction solution was stirred at room temperature for 2 hours. After the reaction was monitored to be complete, the reaction system was purified by Prep-HPLC to afford the preparation solution, then it was freeze-dried to obtain the product of 66 mg, with a yield of 810%. MS calcd for C₄₃H₆₁N₆O₁₉⁺: [M+H]⁺=965.4, found: 965.5.

### Step 3.2 Synthesis of Compound 3b

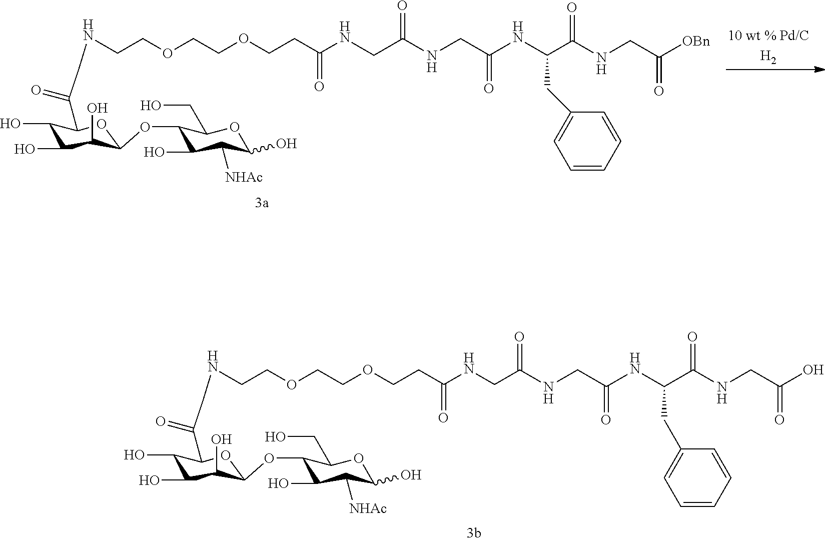

Compound 3a (28 mg) was weighed, added into a single-mouth flask and dissolved by 10 mL of water. 10% Pd/C (2.8 mg, 10% w/w %) was added into the reaction solution, and the gas in the single-mouth flask was replaced with a hydrogen balloon for 3 times, and the reaction solution was stirred at room temperature for 2 hours. After the reaction was monitored to be complete, the reaction solution was filtered, and the freeze-dried filtrate was directly used for the next reaction. MS calcd for C₃₅H₅₅N₆O₁₉⁺: [M+H]⁺=875.3, found: 875.5.

### Step 3.3 Synthesis of Compound 3c

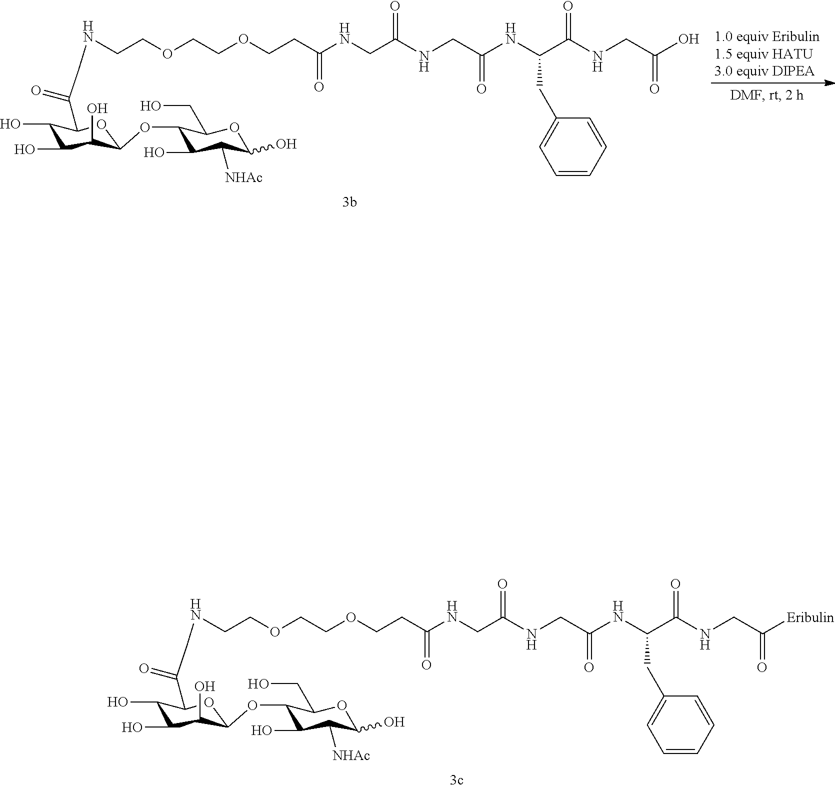

Compound 3b (9.8 mg, 1.1 e.q.) and Eribulin mesylate (8.4 mg, 1.0 e.q.) were weighed, added in an eggplant type flask, and dissolved in 0.5 mL of DMF. HATU (5.8 mg, 1.5 e.q.) and DIPEA (5.5 mg, 3.0 e.q.) were sequentially added and the reaction solution was stirred at room temperature for 2 hours. After the reaction was monitored to be complete, the reaction system was purified by Prep-HPLC to afford the preparation solution. The preparation solution was freeze-dried to obtain the product of 11.5 mg, with a yield of 71%. MS calcd for C₇₆H₁₁₂N₇O₂₉⁺: [M+H]⁺=1586.8, found: 1586.7.

### Step 3.4 Synthesis of Compound 3

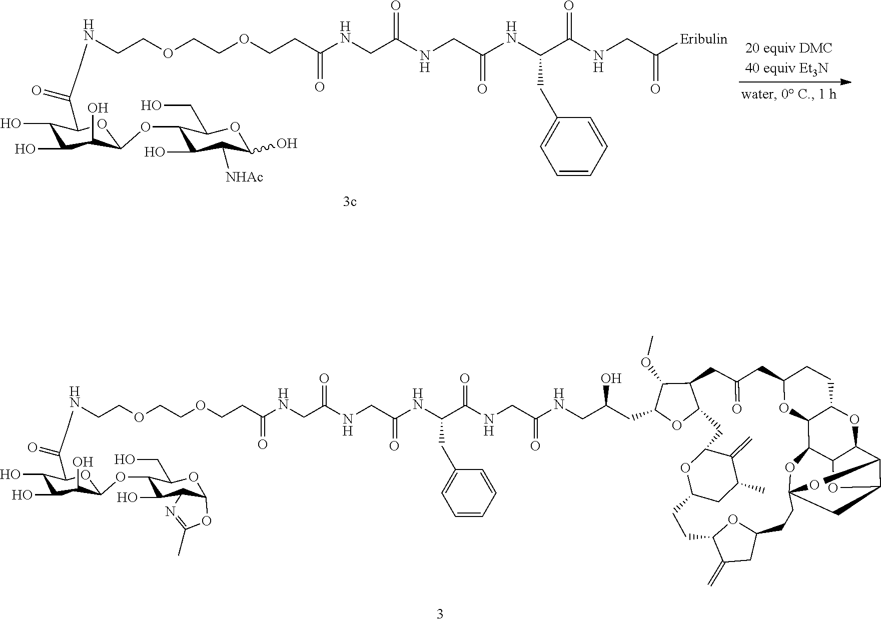

Compound 3c (11.5 mg, 0.007 mmol) was weighed, added in an eggplant type flask, and dissolved in 0.5 mL of water. The reaction solution was cooled to 0-5° C. in an ice bath. Triethylamine (29 mg, 0.29 mmol) and DMC (24.5 mg, 0.145 mmol) were sequentially added, the resulting mixture was stirred at certain temperature (e.g., −20° C. to 40° C.). After the reaction was monitored to be complete, the reaction solution was purified by Prep-HPLC to afford the preparation solution. The preparation solution was freeze-dried to obtain the product of 5.5 mg, with a yield of 50%. MS calcd for C₇₆H₁₁₂N₇O₂₉⁺: [M+H+H₂O]⁺=1586.8, found: 1586.7.

### Example 4: Preparation of Compound 4

The Compound 4 was prepared according to the procedures of Compound 3, wherein PEG4-GGFG-OBn was used instead of PEG2-GGFG-OBn.

MS calcd for C₈₀H₁₂₀N₇O₃₁⁺: [M+H+H₂O]⁺=1674.9, found: 1674.8.

### Example 5: Preparation of Compound 5

### Step 5.1 Preparation of Compound Fmoc-PEG4-VCPAB-Eribulin

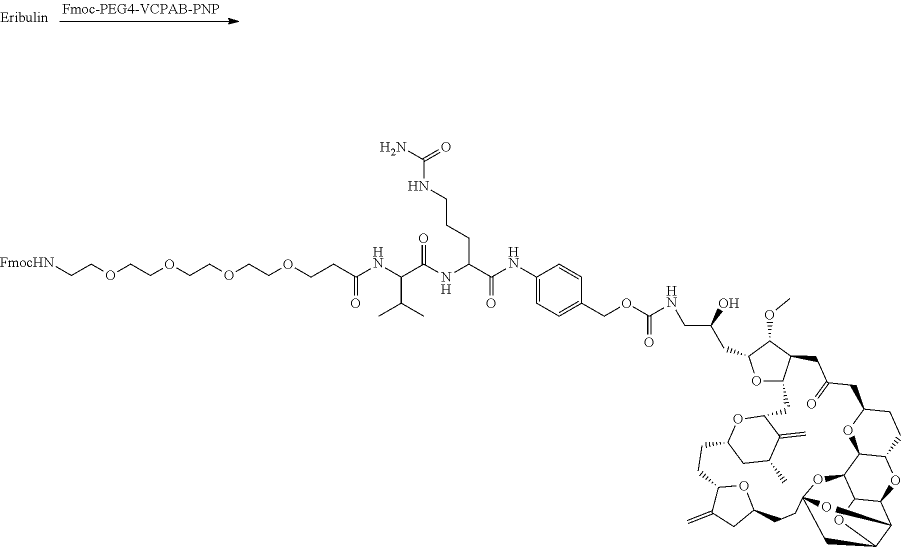

Eribulin mesylate (78.5 mg, 0.095 mmol) and Fmoc-PEG4-VCPAB-PNP (115 mg, 0.113 mmol) were weighed and dissolved in 2.5 mL of DMF at room temperature. HOBT (14.7 mg, 0.095 mmol) and DIPEA (24.5 mg, 0.096 mmol) were sequentially added, then stirred at room temperature for 2 h. After the reaction was monitored to be complete, the mixture was directly used for the next step.

### Step 5.2 Preparation of Compound PEG4-VCPAB-Eribulin

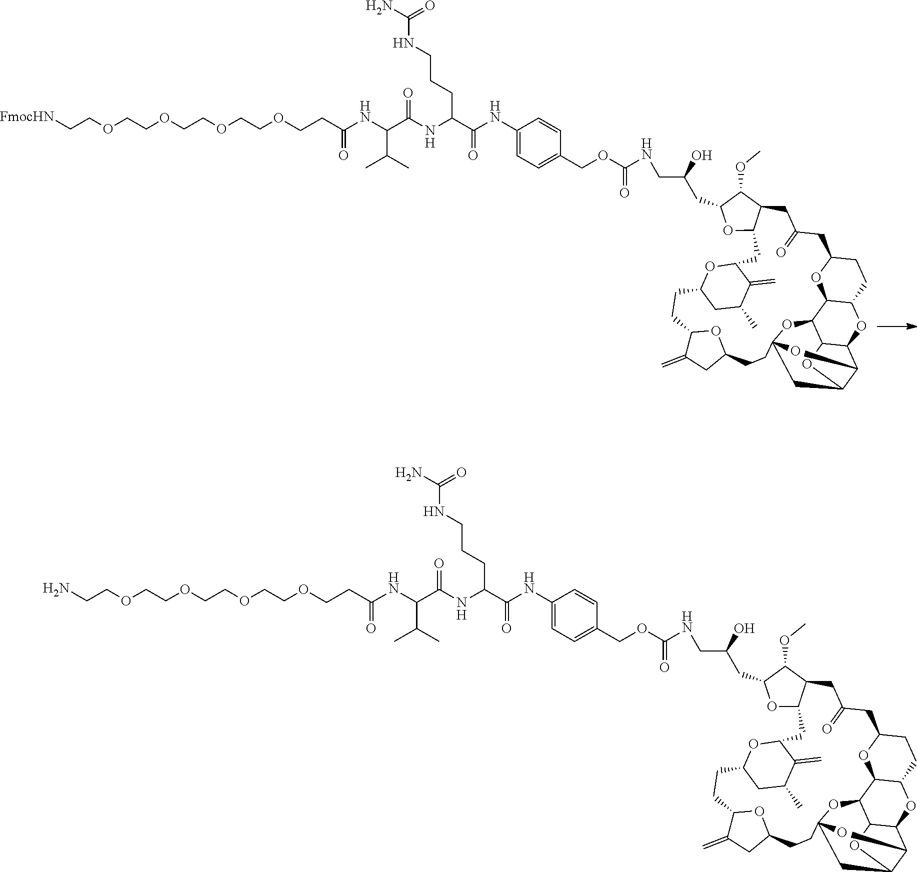

200 μL of diethylamine was added to the reaction solution of the previous step, then stirred at room temperature for 30 min. After the reaction was monitored to be complete, the reaction was neutralized with 30% HOAc, and then purified by Prep-HPLC to afford the preparation solution. The preparation solution was freeze-dried to obtain the product of 64 mg, with a yield of 50% in two steps. MS calcd for C₇₀H₁₀₈N₇O₂₁⁺: [M+H]⁺=1382.7, found: 1382.6.

### Step 5.3 Preparation of Compound 5a

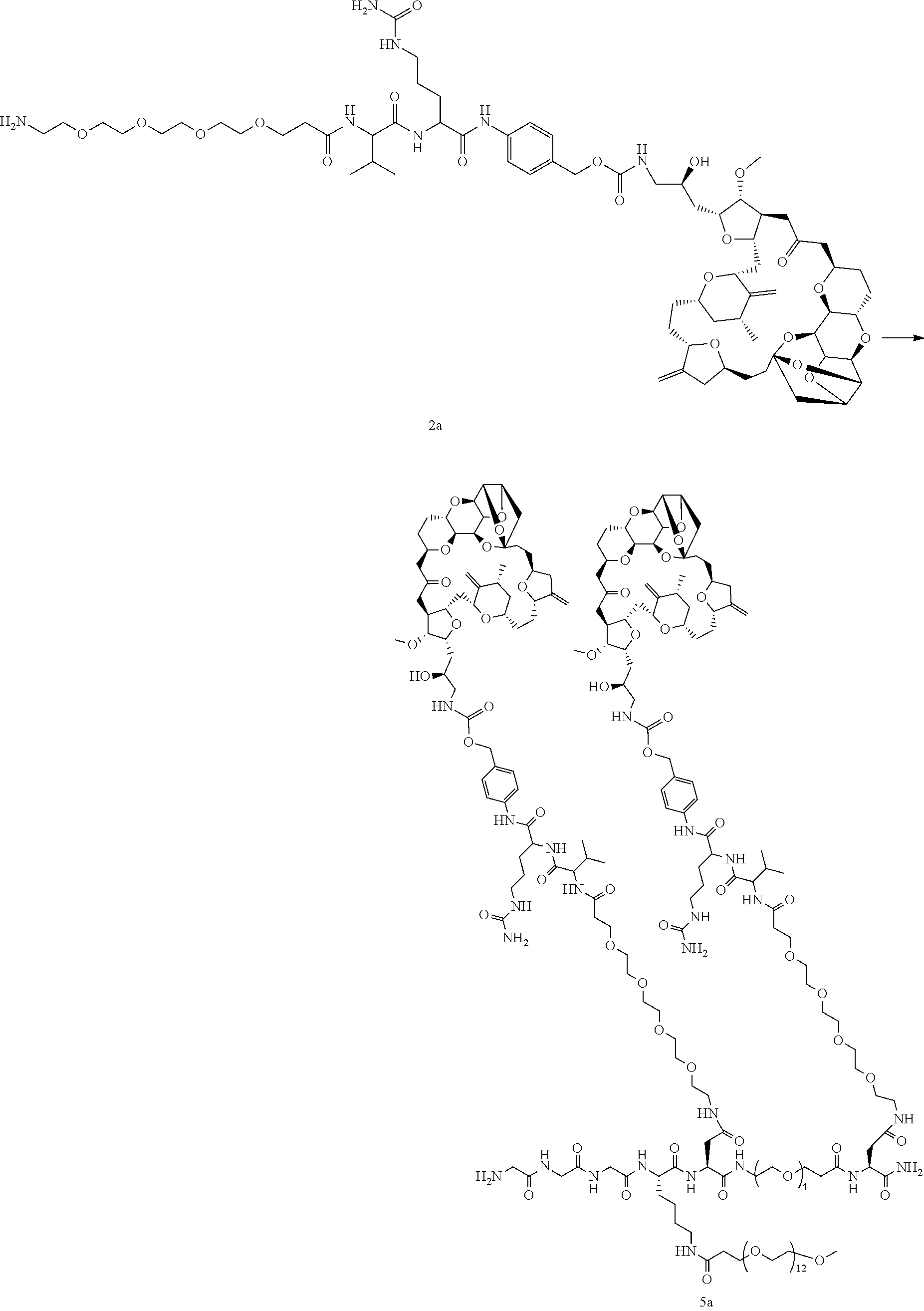

Compound 2a (37.7 mg, 0.023 mmol) and PEG 4-VCPAB-Eribulin (64 mg, 0.046 mmol) were weighed, added into a single-mouth flask, and dissolved in DMF (20 mL) at room temperature. DIPEA (11.9 mg, 0.092 mmol) and HATU (21 mg, 0.055 mmol) were sequentially added, and the reaction solution was stirred at room temperature for 2 hours. After the reaction was monitored to be complete, the reaction solution was directly used for the next step.

### Step 5.4 Preparation of Compound 5b

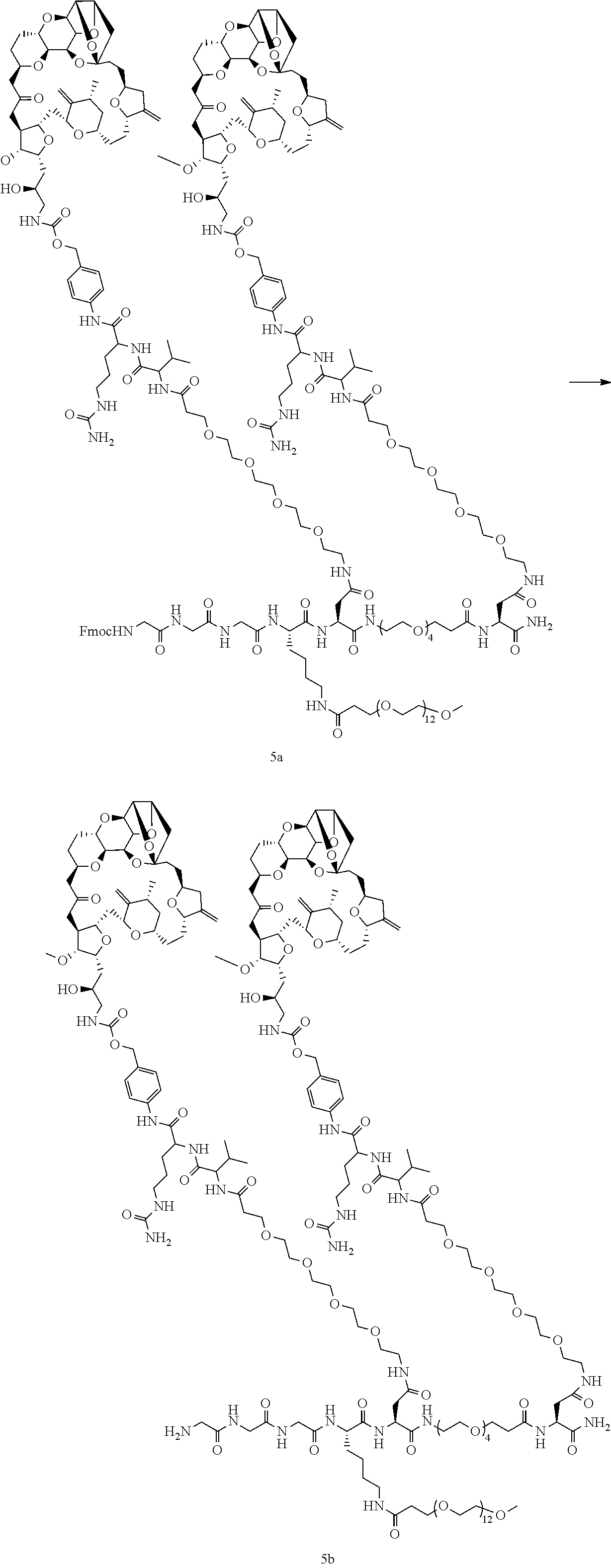

200 μL of diethylamine was added to the reaction solution of the previous step, then stirred at room temperature for 30 min. After the reaction was monitored to be complete, the reaction was neutralized with 30% HOAc, and then purified by Prep-HPLC to afford the preparation solution. The preparation solution was freeze-dried to obtain the product of 33.8 mg, with a yield of 35% in two steps. MS Calcd for C₁₉₉H₃₂₃N₂₃O₆₉⁴⁺ [(M+4H)/4]⁺=1034.8, found: 1035.0.

### Step 5.5 Preparation of Compound 5c

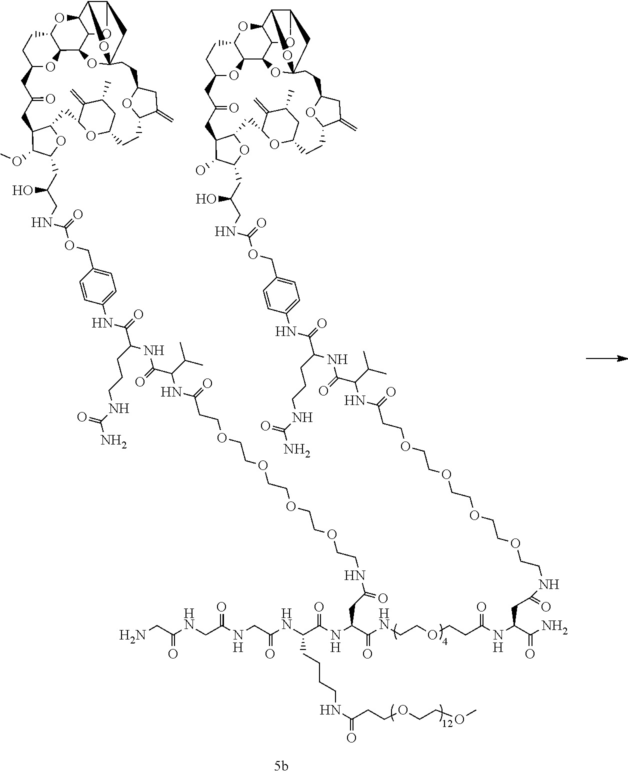

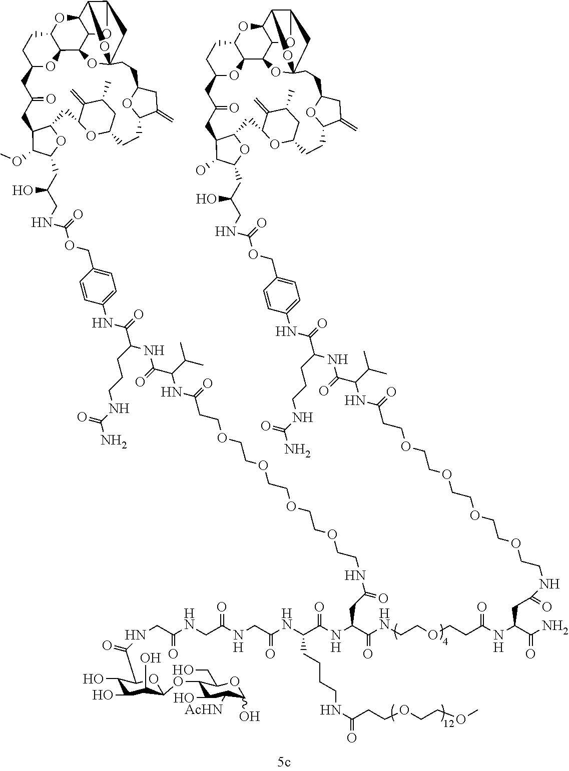

Compound 5b (34 mg, 0.008 mmol) and Compound 1 (6.2 mg, 0.016 mmol) were weighed, added into a single-mouth flask, and dissolved in DMF (5 mL) at room temperature. DIPEA (2.6 mg, 0.020 mmol) and HATU (4.7 mg, 0.012 mmol) were sequentially added and the reaction solution was stirred at room temperature for 2 hours. After the reaction was monitored to be complete, the reaction solution was purified by Prep-HPLC to obtain the product of 33 mg, with a yield of 91%. MS calcd for C₂₁₃H₃₄₃N₂₄O₈₀³⁺ [(M+3H)/3]⁺=1505.8, found: 1505.6.

### Step 5.6 Preparation of Compound 5

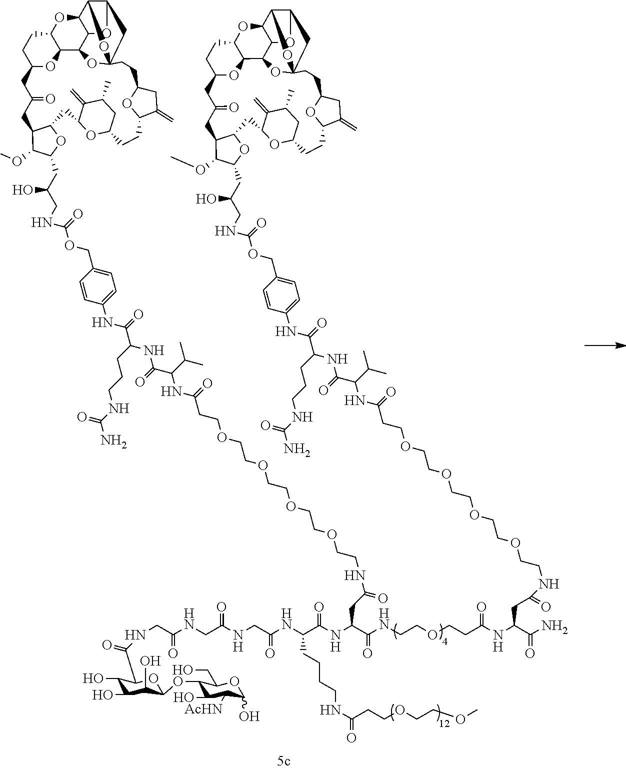

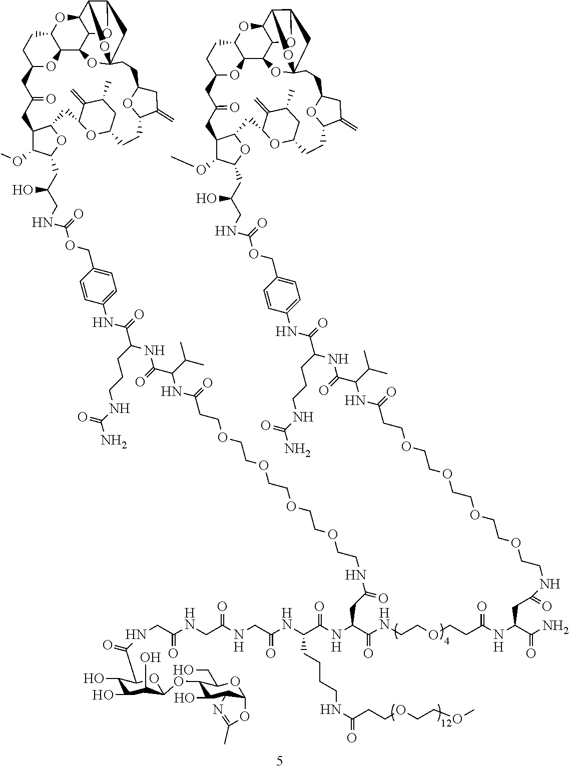

Compound 5c (33 mg, 0.0073 mmol) was dissolved in 5 mL of H₂O. DMC (37 mg, 0.219 mmol) and TEA (44 mg, 0.438 mmol) were sequentially added at 0° C., and the reaction solution was stirred at 0° C. for 1 h. After the reaction was monitored to be complete, the reaction solution was purified by Prep-HPLC to afford the preparation solution. The preparation solution was freeze-dried to obtain the product of 21 mg, with a yield of 63%. LCMS: Calcd for C₂₁₃H₃₄₃N₂₄O₈₀³⁺ [(M+3H⁺H₂O)/3]⁺: 1505.8, found: 1505.60.

### Example 6: Preparation of Antibodies

Production, purification, and characterization of anti-human ErbB2/Her2 antibody T-LCCT_{L}-HC are referenced to the Example 1 of US 20170112944 A1, which is incorporated herein by reference in its entirety.

### Example 7: Preparation and the DAR Detection of ADCs

### General Preparation Protocol for the ADCs

Preparation of ADC drug was based on the glycan-remodeling conjugation of the antibody with linker-payload mediated by the immobilized endoglycosidase catalysis. In 1× endoglycosidase buffer, the antibody was mixed thoroughly with linker-payload in an appropriate molar ratio (1:1 to 1:100), then the immobilized endoglycosidase medium was added and mixed. The conjugation reaction in the mixed suspension was carried out at certain temperature (4-40° C.) for a certain time (0.5-20 h). Upon completion, the solid phase conjugation mixture was centrifuged, purified, ultrafiltered or dialyzed to remove unreacted drug intermediates. Purified ADC drug was stored in 1×PBS pH 7.4 at 2-8° C. or −80° C.

ADC-1, ADC-2, ADC-3 and ADC-4 are prepared according to the above protocol, wherein the linker-payload compound of Compound 2, Compound 3, Compound 4 and Compound 5 were used respectively to conjugate with the antibody prepared in Example 6.

ADC-5 is prepared according to the traditional chemical conjugation method, which is described below:

The antibody T-LCCT_{L}-HC and TCEP (molar ratio of 1:1-1:20) were added into PBS buffer, and the reaction mixture was reacted at 2-40° C. for 2 hours. Compound 6 (MAL-PEG2-Val-Cit-PAB-Eribulin, 1 e.q. −100 e.q. based on the amount of antibody, commercially available, and the structure can be found in CN114272389B) was dissolved in DMSO and added to the antibody, and the reaction continued at 4-40° C. for 0.5-20 hours. Then cysteine (1-20 e.q. based on the amount of antibody) and DHAA (1-20 e.q. based on the amount of antibody) were added into the reaction and the mixture was continued to react. The resulting ADC (ADC-5) was purified, ultrafiltered or dialyzed to remove unreacted drug intermediates. The purified ADC-5 was stored in 1×PBS pH 7.4 at 4° C. or −80° C.

The DAR distribution of the ADCs was determined by the HIC-HPLC, and the results are shown below:

| ADCs | DAR |
|---|---|
| ADC-1 | 3.82 |
| ADC-2 | 1.76 |
| ADC-3 | 1.81 |
| ADC-4 | 3.86 |
| ADC-5 | 3.79 |

### Example 8: In Vitro Activity Assay of ADCs

The inhibitory effect of ADC on tumor cell proliferation was evaluated in human tumor cell lines with different HER2 expression levels (HER2 high expression tumor cells: SK-BR-3 and NCI-N87; HER2-negative tumor cell: MDA-MB-468). Tumor cells in the logarithmic phase of growth were collected, and 5000 cells per well were evenly inoculated in each hole of the 96-well plate. After the cell adhesion overnight, the cells were treated with different concentrations of drugs for 96 hours. Cell viability was measured by CellTiter-Glo® luminescence cell viability detection kit, and the percentage of cell viability was calculated.

TABLE 1
The inhibitory effects of ADCs on SK-BR-3 tumor cells

| Drug | IC₅₀ (nM) | R² |
|---|---|---|
| ADC-1 | 0.00727 | 0.9933 |
| ADC-3 | 0.0115 | 0.9885 |
| ADC-4 | 0.01178 | 0.9988 |
| ADC-5 | 0.0104 | 0.9962 |
| Eribulin(commercially | 0.3052 | 0.9994 |
| available) | | |

TABLE 2
The inhibitory effects of ADCs on NCI-N87 tumor cells

| Drug | IC₅₀ (nM) | R² |
|---|---|---|
| ADC-1 | 0.02841 | 0.9948 |
| ADC-3 | 0.05077 | 0.9968 |
| ADC-4 | 0.02518 | 0.9974 |
| ADC-5 | 0.027 | 0.9978 |
| Eribulin(commercially | 0.6138 | 0.9957 |
| available) | | |

TABLE 3
The inhibitory effects of ADCs on MDA-MB-468 tumor cells

| Drug | IC₅₀ (nM) | R² |
|---|---|---|
| ADC-1 | N/A | N/A |
| ADC-3 | N/A | N/A |
| ADC-4 | N/A | N/A |
| ADC-5 | N/A | N/A |
| Eribulin (commercially | 0.09329 | 0.9551 |
| available) | | |

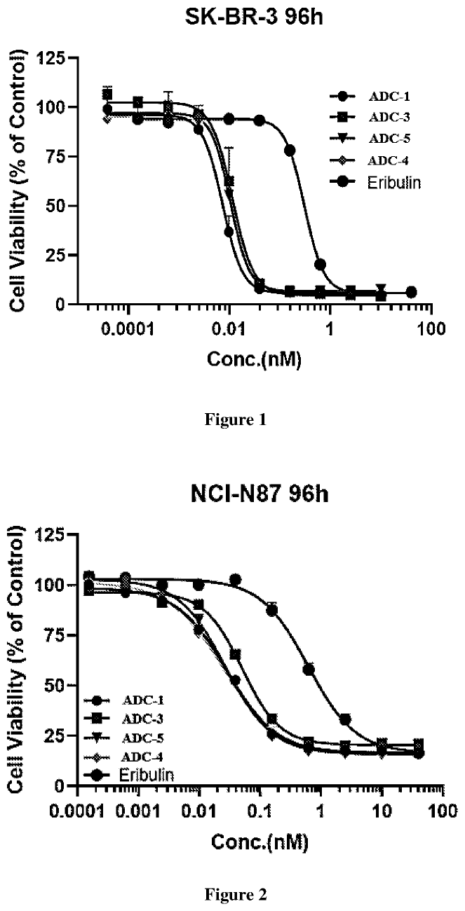

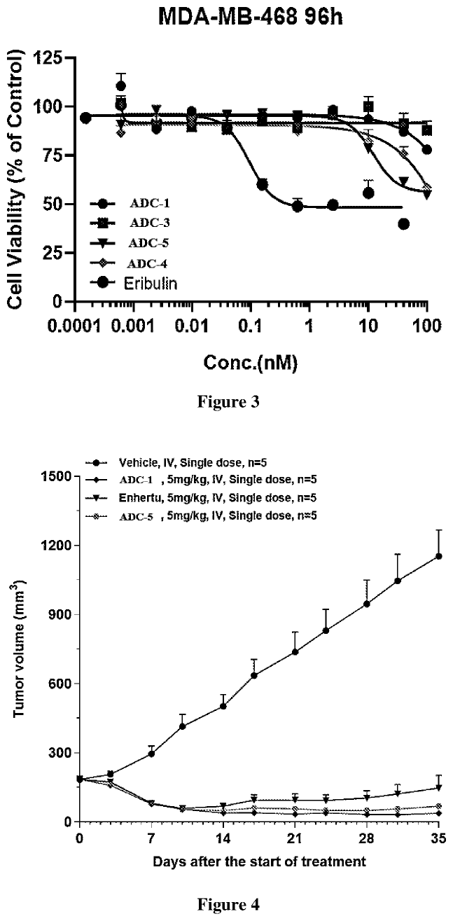

The results are as shown in Tables 1-3 and FIGS. 1-3, respectively. The results indicates that all ADC drugs show obvious inhibitory effects on the proliferation of tumor cells with high expression of HER2 NCI-N87 and SK-BR-3, and they are better than Eribulin. There is no significant difference in the inhibitory ability of ADC with the same DAR value on the proliferation of positive tumor cells. The inhibitory ability of ADC-1 on the proliferation of positive cells is slightly better than that of ADC-3, which is related to the different toxin contents carried by them. For HER2-negative MDA-MB-468 tumor cells, neither ADC-1 nor ADC-3 shows obvious cytotoxicity, which indicates that ADCs are stable, with less payload shedding and high safety. However, ADC-4 and ADC-5 have obvious cytotoxicity to negative cells (see FIG. 3), which may be due to the shedding of small molecules, which leads to nonspecific cell killing.

### Example 9: In Vivo Activity Assay of ADCs

NCI-N87 human gastric cancer cells were subcutaneously inoculated on the right back of female BALB/c nude mice. On the 6th day after tumor cell inoculation, the tumor-bearing mice were randomly divided into 4 groups, with 5 mice in each group. On the day of grouping, tumor-bearing mice were given 5 mg/kg of ADC-1, ADC-5 and Enhertu (commercially available), respectively. The diameter of tumor was measured with vernier caliper twice a week. The calculation formula of tumor volume is V=0.5a×b², wherein a and b represent the long and short diameters of tumor respectively. The weight of mice was measured twice a week, and the behavior, food intake, physical signs or other abnormal behaviors were observed every day.

The calculation formula of relative tumor proliferation rate T/C (%) is as follows: T/C %=TRTV/CRTV×100% (TRTV: treatment group RTV; CRTV: negative control group RTV; RTV: relative tumor volume). RTV=Vₜ/V₀, where V₀ is the average tumor volume measured at the time of group administration, Vₜ is the average tumor volume measured at a certain time, and TRTV and CRTV take the data on the same day.

The calculation formula of tumor growth inhibition rate TGI (%) is as follows: TGI (%)=[1−(Tᵢ−T₀)/(Vᵢ−V₀)]×100, wherein Tᵢ is the average tumor volume of the treatment group on a certain day, T₀ is the average tumor volume of the treatment group at the beginning of drug administration, Vᵢ is the average tumor volume of the vehicle control group on the same day as Tᵢ, and V₀ is the average tumor volume of the vehicle control group at the beginning of treatment.

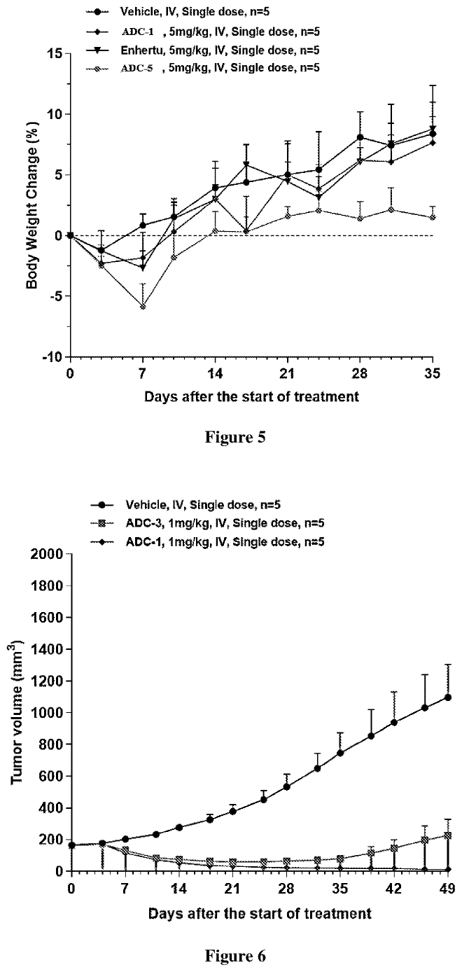

The changes of tumor volume and weight in different treatment groups at different time points after the start of treatment are shown in the FIGS. 4-5. 35 days after administration, compared with the vehicle control group, ADC-1, ADC-5 and Enhertu all have significant anti-tumor effects at the dose of 5 mg/kg. The antitumor effect of ADC-1 is slightly better than that of ADC-5, and obviously better than that of Enhertu.

ADC-5 has a great influence on the weight of mice. On the seventh day after administration, the weight loss of mice in ADC-5 group is significantly higher than that in other groups. From 21 days to 35 days after administration, the weight of mice in ADC-5 group does not increase, and is substantially in a flat state, while the weight of mice in other groups continue to increase.

Capan-1 human pancreatic cancer cells were subcutaneously inoculated on the right back of female BALB/c nude mice. On the 7th day after tumor cell inoculation, the tumor-bearing mice were randomly divided into 3 groups, with 5 mice in each group. On the day of grouping, tumor-bearing mice were given 1 mg/kg of PBS (Vehicle), ADC-1 and ADC-3, respectively. The diameter of tumor was measured with vernier caliper twice a week. The calculation formula of tumor volume is V=0.5a×b², wherein a and b represent the long and short diameters of tumor respectively. The weight of mice was measured twice a week, and the behavior, food intake, physical signs or other abnormal behaviors were observed every day.

The calculation formula of relative tumor proliferation rate T/C (%) is as follows: T/C %=TRTV/CRTV×100% (TRTV: treatment group RTV; CRTV: negative control group RTV; RTV: relative tumor volume). RTV=Vₜ/V₀, where V₀ is the average tumor volume measured at the time of group administration, Vₜ is the average tumor volume measured at a certain time, and TRTV and CRTV take the data on the same day.

The calculation formula of tumor growth inhibition rate TGI (%) is as follows: TGI (%)=[1−(Tᵢ−T₀)/(Vᵢ−V₀)]×100, wherein Tᵢ is the average tumor volume of the treatment group on a certain day, T₀ is the average tumor volume of the treatment group at the beginning of drug administration, Vᵢ is the average tumor volume of the vehicle control group on the same day as Tᵢ, and V₀ is the average tumor volume of the vehicle control group at the beginning of treatment.

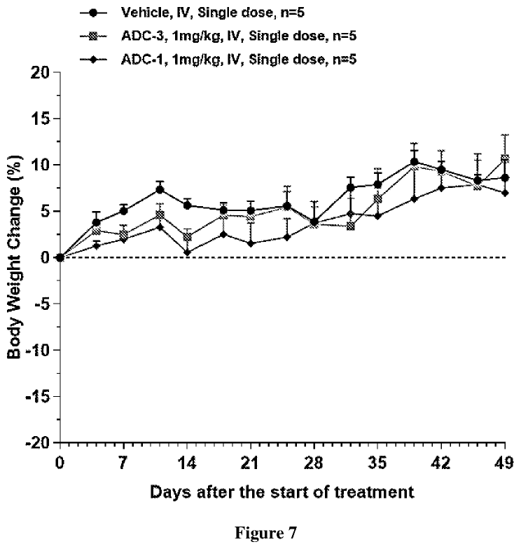

The changes of tumor volume and weight in different treatment groups at different time points after the start of treatment are shown in the FIGS. 6-7. 49 days after administration, compared with the vehicle control group, ADC-1 and ADC-3 both shows significant anti-tumor effects at the dose of 1 mg/kg. The antitumor effect of ADC-1 is slightly better than that of ADC-3. There is no obvious difference in body weight among all 3 groups.

### Example 10: Tolerance Study in Mice of ADCs

For the first run of study, 6 male C57BL/6 mice were randomly assigned into 2 groups and given ADC-1 at 40 and 80 mg/kg on Day 1, respectively. For the second run of study, 16 C57BL/6 mice (8 males and 8 females) were randomly assigned into 4 groups. Group 1 were given saline once every two weeks for a total of 2 repeated doses; Group 2 were given ADC-5 at 36 mg/kg on Day 1 and 80 mg/kg on Day 15; Group 3 were given ADC-4 at 44 mg/kg on Day 1 and 80 mg/kg on Day 15; Group 4 were given ADC-1 at 120 mg/kg on Day 1 and Day 15. During the above 2 studies, clinical observation, body weight, food consumption and gross pathology were evaluated.

### Clinical Observation and Gross Pathology

ADC-5: One male mouse showed rapid breathing, low temperature and low motility on Day 25 (10 days after dosed at 80 mg/kg), and was found died on Day 26. Result of gross pathology showed discoloration of liver, kidney and spleen, collapse of abdominal vessels, 0.6-0.7 mL pleural effusion (dark red liquid), small heart, discoloration of lungs (mottled, multiple, dark red).
ADC-4: No any clinical observation abnormality was noted, however, result of gross pathology shows small liver and discoloration of lungs (mottled, multiple, dark red) in all 4 mice.
ADC-1: No any clinical observation abnormality was noted after doses of 40 mg/kg and 80 mg/kg. One female mouse showed moderate dehydration, left torticollis, mobility impairment, and rotation on Day 8 after the first dose of 120 mg/kg, and then was euthanized. Result of gross pathology shows empty digestive tract and rough surface, discolored (red and gray), and hard texture in livers. One female mouse was found died on Day 22 (7 days after the second dose of 120 mg/kg), result of gross pathology shows empty digestive tract, large size, discolored (mottled, dark red) in lungs, and large spleen.

### Food Consumption and Body Weight

ADC-5: No abnormity was observed in food consumption and body weight after dosed of 36 mg/kg, but significantly lower food consumption and body weight was noted both in males and females after dosed of 80 mg/kg.
ADC-4: No abnormity was observed in food consumption and body weight after dosed of 44 mg/kg, but significantly lower food consumption and body weight was noted both in males and females after dosed of 80 mg/kg.
ADC-1: No abnormity was observed in food consumption and body weight after dosed of 40 mg/kg and 80 mg/kg, but significantly lower food consumption and body weight was noted both in males and females after dosed of 80 mg/kg.

### CONCLUSION

Under the conditions of this study, the maximum tolerated dose (MTD) of ADC-5 is 36-80 mg/kg, the MTD of ADC-4 is about 80 mg/kg, and the MTD of ADC-1 is 80-120 mg/kg.

Sequence listing

SEQ ID No. 1: T-LCCT_{L}-HC Light chain:
DIQMTQSPSSLSASVGDRVTITCRASQDVNTAVAWYQQKPGKAPKLLIY
SASFLYSGVPSRFSGSRSGTDFTLTISSLQPEDFATYYCQQHYTTPPTF
GQGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGECGALPETGG
SEQ ID No. 2: T-LCCT_{L}-HC Heavy chain:
EVQLVESGGGLVQPGGSLRLSCAASGFNIKDTYIHWVRQAPGKGLEWVA
RIYPTNGYTRYADSVKGRFTISADTSKNTAYLQMNSLRAEDTAVYYCSR
WGGDGFYAMDYWGQGTLVTVSSASTKGPSVFPLAPSSKSTSGGTAALGC
LVKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVFL
FPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAK
GQPREPQVYTLPPSREEMTKNQVSLTCLVKGFYPSDIAVEWESNGQPEN
NYKTTPPVLDSDGSFFLYSKLTVDKSRWQQGNVFSCSVMHEALHNHYTQ
KSLSLSPGK
SEQ ID No. 3: GGGGS
SEQ ID No. 4: GGGGSGGGGS
SEQ ID No. 5: GGGGSGGGGSGGGGS
SEQ ID No. 6: GGFG
SEQ ID No. 7: GFLG
SEQ ID No. 8: ALAL

1. A linker-payload compound, which has the structure of formula (I): 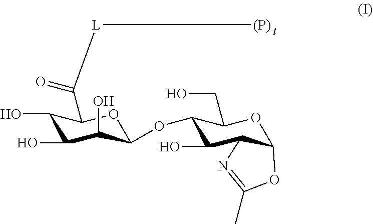
wherein
L is a connecting moiety comprising —NH—, and L is directly connected with the carbonyl group in the disaccharide moiety via the —NH—,
wherein
when L is a linear connecting moiety, it is connected with one P moiety, and t is 1; or
when L is a branched connecting moiety, each of the branches is connected with one P moiety at most, and t is an integer greater than 1 (for example, 2, 3, 4, 5, 6, 7, 8, 9 or 10);
P is a payload with the structure of 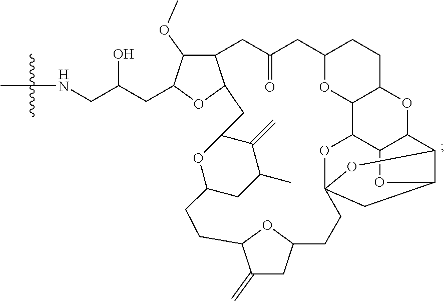 [SMILES: *NCC(O)CC1OC2CC3OC(CCC4OC(CCC56CC7OC8C(OC9CCC(CC(=O)CC2C1OC)OC9C8O5)C7O6)CC4=C)CC(C)C3=C]
preferably, P is Eribulin with the structure of

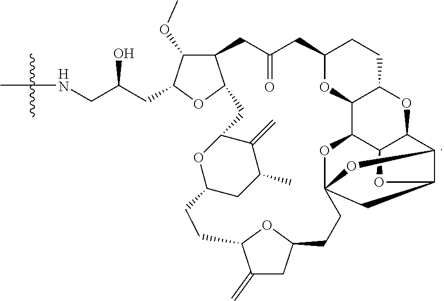 [SMILES: *NC[C@@H](O)C[C@H]1O[C@H]2C[C@H]3O[C@@H](CC[C@@H]4O[C@@H](CC[C@@]56C[C@H]7O[C@H]8[C@@H](O5)[C@H]5O[C@H](CC[C@@H]5O[C@H]8[C@H]7O6)CC(=O)C[C@@H]2[C@H]1OC)CC4=C)C[C@@H](C)C3=C]

2. The linker-payload compound according to claim 1, wherein
-L- is -L₂-L₁- and t is 1, and
the compound of formula (I) has the structure of formula (I-1): 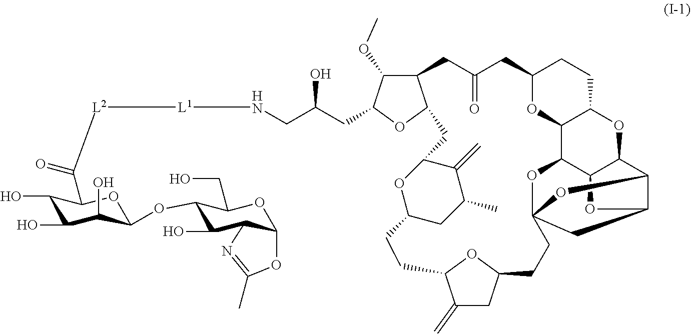
wherein,
L¹ comprises a cleavable sequence containing an amino acid sequence that is enzymatically cleavable, wherein the amino acid sequence contains 1, 2, 3, 4, 5, 6, 7, 8, 9 or 10 amino acids;
L² comprises an amino acid residue sequence of -*(AA)ₙ**-, wherein n is an integer of 1-100, AA is an amino acid residue independently at each occurrence, * represents the N-terminal of the corresponding amino acid, ** represents the C-terminal of the corresponding amino acid, and —(C₂H₄—O)ₘ—(CH₂)ₚ— is optionally present between the amino group and the α-carbon of an amino acid, wherein m is 1, 2, 3, 4, 5, 6, 7, 8, 9 or 10; p is 0, 1, 2 or 3, and the * terminal forms an amide bond with the carbonyl group in the disaccharide moiety.

3. The linker-payload compound according to claim 2, wherein AA is independently any one of Phe, Lys, Gly, Ala, Leu, Asn, Val, Ile, Pro, Trp, Ser, Tyr, Cys, Met, Asp, Gln, Glu, Thr, Arg and His or any combination thereof at each occurrence.

4. The linker-payload compound according to claim 2, wherein n is an integer of 1-50, preferably an integer of 1-30, preferably an integer of 1-20, preferably an integer of 1-10, preferably 1, 2, 3, 4, 5, 6, 7, 8, 9 and 10.

5. The linker-payload compound according to claim 2, wherein the amino acid sequence that is enzymatically cleavable is -Val-Ala-, -Gly-Gly-Phe-Gly-, -Phe-Lys-, -Val-Cit-, -Val-Lys-, -Gly-Phe-Leu-Gly-, -Ala-Leu-Ala-Leu- and -Ala-Ala-Ala- or a combination thereof.

6. The linker-payload compound according to claim 2, wherein L¹ further contains a self-immolative spacer, preferably, the self-immolative spacer is PABC.

7. The linker-payload compound according to claim 2, wherein L¹ is -Gly-Gly-Phe-Gly-.

8. The linker-payload compound according to claim 2, which is as the structure below: 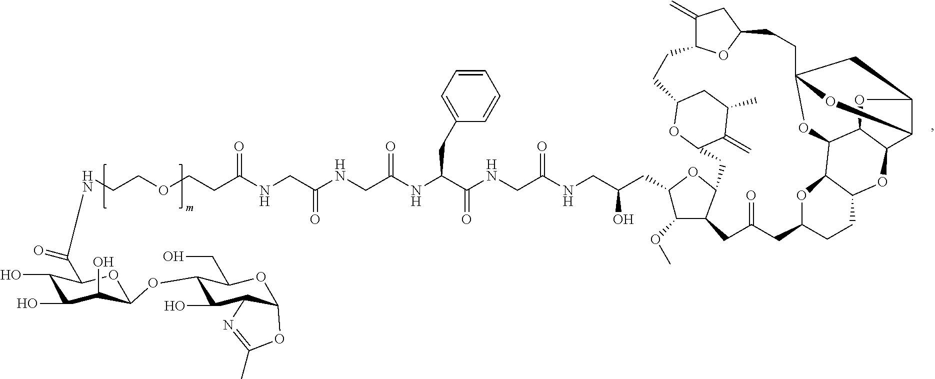
wherein m is an integer of 1, 2, 3, 4, 5, 6, 7, 8, 9 and 10; preferably an integer of 1, 2, 3, 4 or 5.

9. The linker-payload compound according to claim 2, which is selected from:

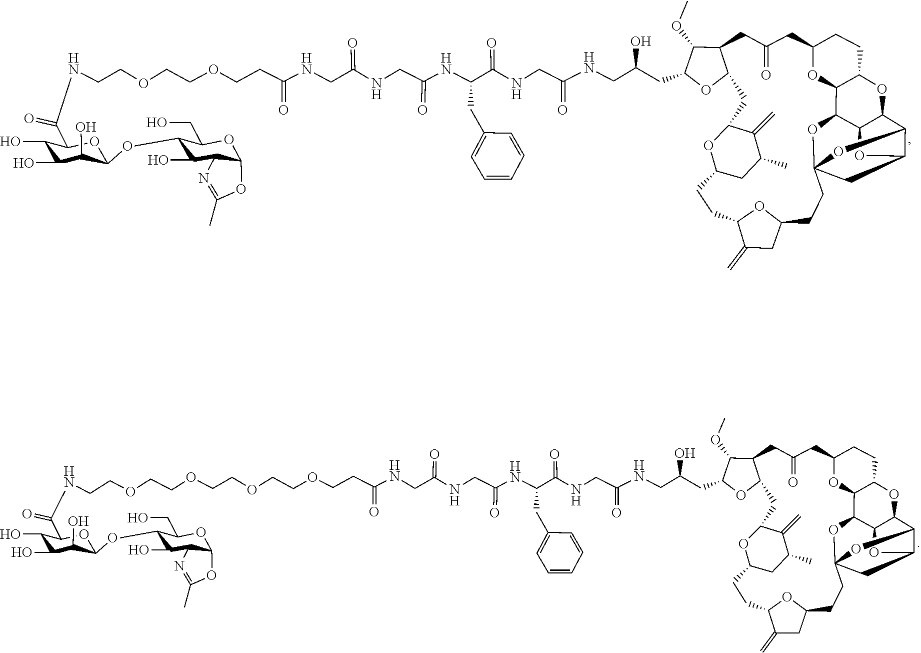

10. The linker-payload compound according to claim 1, wherein the compound of formula (I) has the structure of formula (I-2): 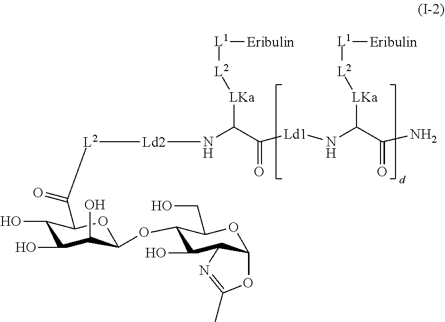
wherein,
Ld2 is independently a bond, or a natural amino acid or an oligomeric natural amino acid with a polymerization degree of 2-10 (i.e. 2, 3, 4, 5, 6, 7, 8, 9 or 10), wherein the side chain of the natural amino acid or the side chain of the oligomeric natural amino acid is independently unsubstituted or substituted by —CO—(PEG)ⱼ-R¹¹;
Ld1 is independently a bond or —NH—(PEG)ᵢ-(CO)—;
-(PEG)ᵢ- and -(PEG)ⱼ- are each independently a PEG fragment, wherein PEG represents —(O—C₂H₄)— or —(C₂H₄—O)—, with an optional C₁₋₁₀ alkylene at one end;
each Lka is independently selected from 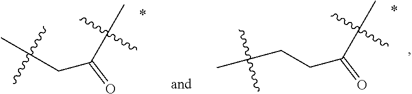 [SMILES: *CC(*)=O ; *CCC(*)=O]
where * represents the connection site with L²;
wherein L² and L¹ are as defined in claim 2; or -L¹- represents -Val-Cit-PABC- or -Gly-Gly-Phe-Gly-;
R¹¹ is C₁₋₁₀ alkyl;
d is 0, 1, 2, 3, 4, 5 or 6;
each i is independently an integer from 1 to 100 (for example, 1, 2, 3, 4, 5, 6, 7, 8, 9 or 10);
each j is independently an integer from 1 to 100 (for example, 1, 2, 3, 4, 5, 6, 7, 8, 9, 10, 11 or 12).

11. The linker-payload compound according to claim 10, which is selected from 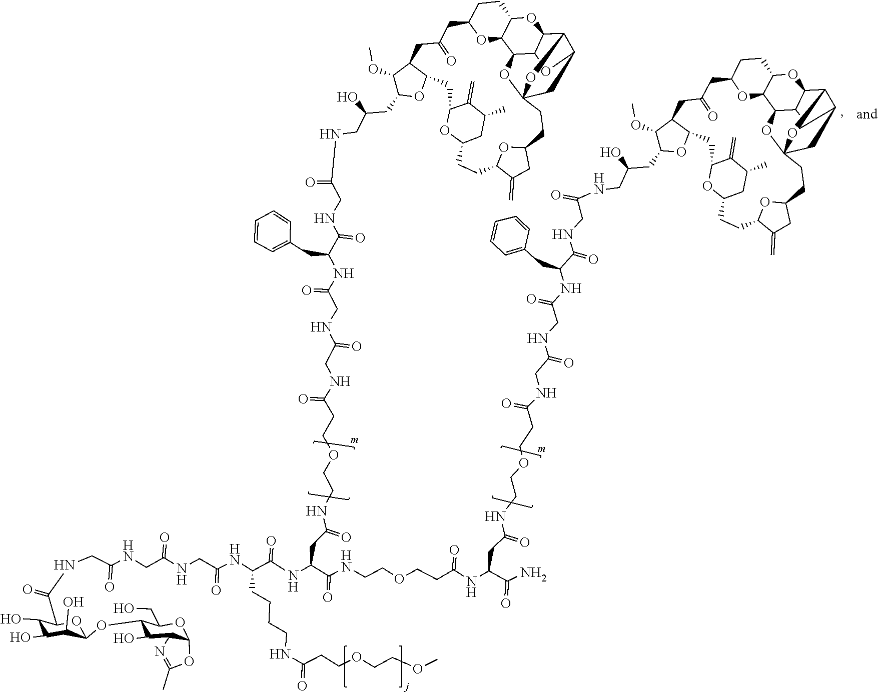 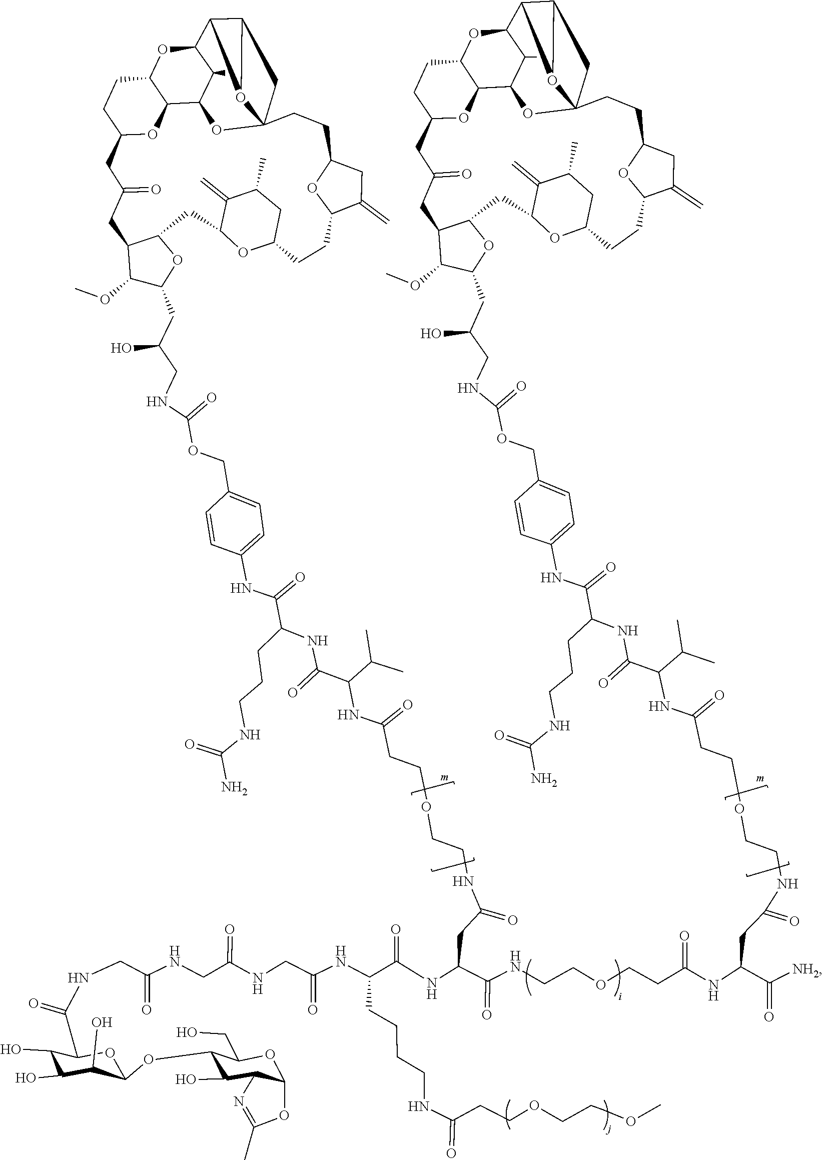
wherein,
each m is independently an integer of 1, 2, 3, 4, 5, 6, 7, 8, 9 and 10; preferably an integer of 1, 2, 3 or 4;
i is independently an integer from 1 to 100 (for example, 1, 2, 3, 4, 5, 6, 7, 8, 9, 10, 11 or 12);
j is independently an integer from 1 to 100 (for example, 1, 2, 3, 4, 5, 6, 7, 8, 9, 10, 11 or 12).

12. The linker-payload compound according to claim 11, which is selected from

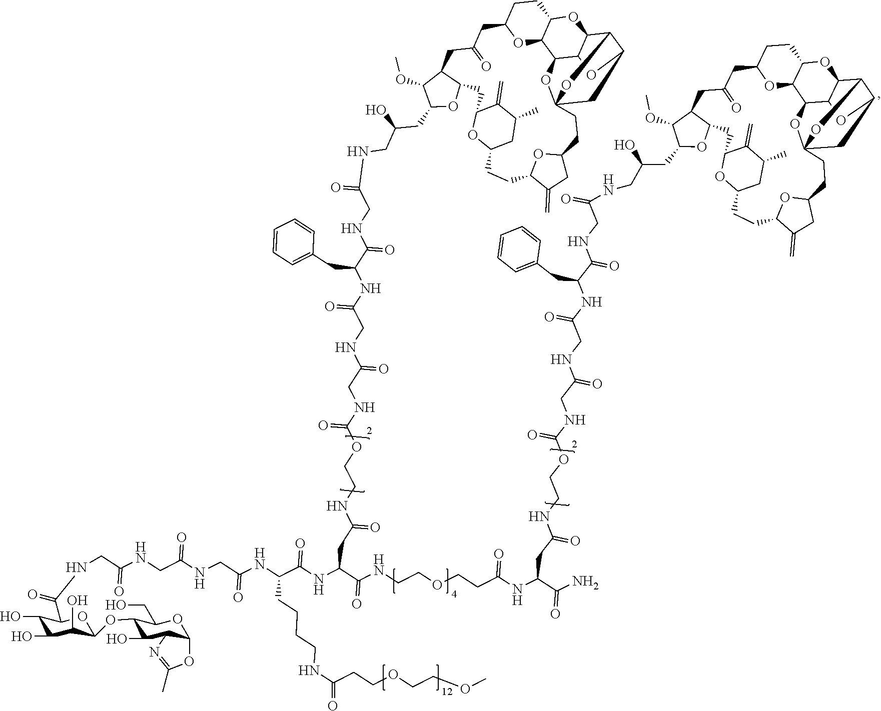

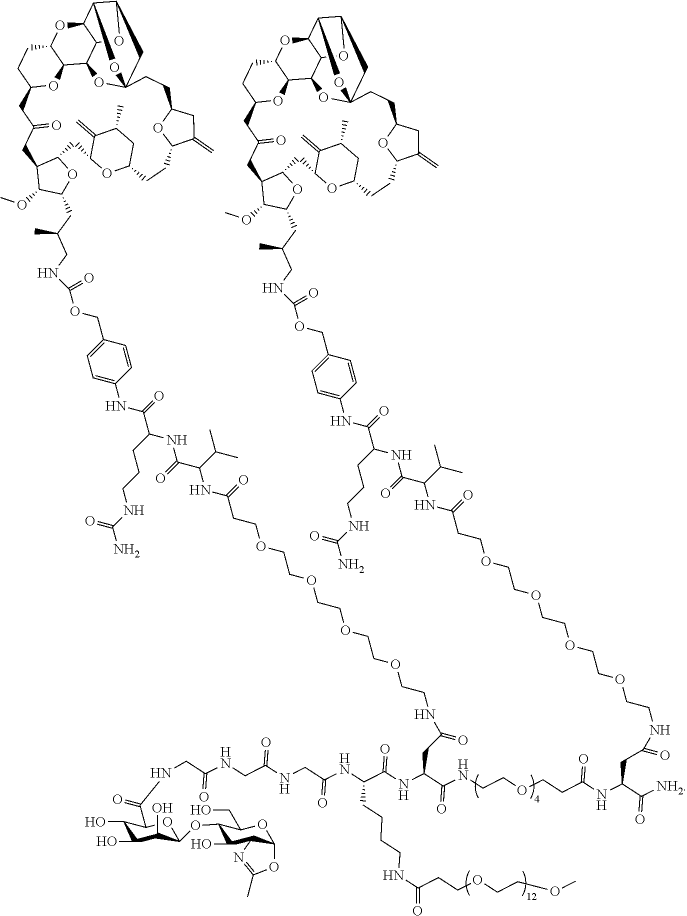

13. A process for preparing the linker-payload compound according to claim 1, which comprises the step of amide formation reaction between 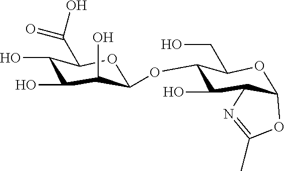
and L′-(P)ₜ to form 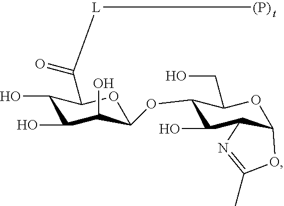
wherein L′ is as defined for L in any one of claims 1 to 10, except that —NH— in L connected to 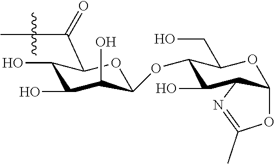
is H₂N— in L′.

14. An antibody-drug conjugate, which has the structure of formula (II): 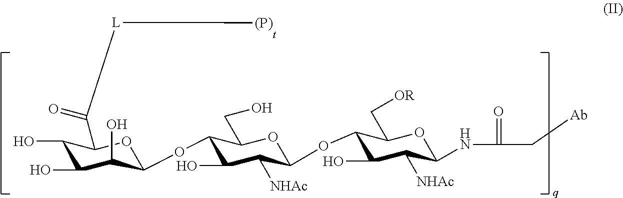
wherein
R is hydrogen or α-L-fucosyl;
q is 1 or 2;
Ab is an antibody or antigen-binding fragment;
L is a connecting moiety comprising —NH—, and L is directly connected with the carbonyl group in the disaccharide moiety via the —NH—,
wherein when L is a linear connecting moiety, it is connected with one P moiety, and t is 1; or when L is a branched connecting moiety, each of the branches is connected with one P moiety at most, and t is an integer greater than 1 (for example, 2, 3, 4, 5, 6, 7, 8, 9 or 10);
P is a payload with the structure of 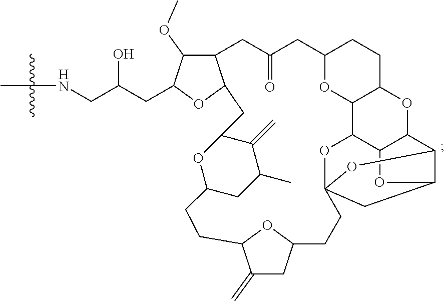 [SMILES: *NCC(O)CC1OC2CC3OC(CCC4OC(CCC56CC7OC8C(OC9CCC(CC(=O)CC2C1OC)OC9C8O5)C7O6)CC4=C)CC(C)C3=C]
preferably, P is Eribulin with the structure of

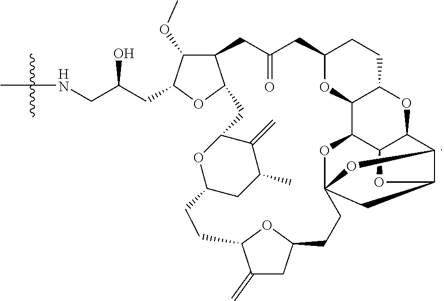 [SMILES: *NC[C@@H](O)C[C@H]1O[C@H]2C[C@H]3O[C@@H](CC[C@@H]4O[C@@H](CC[C@@]56C[C@H]7O[C@H]8[C@@H](O5)[C@H]5O[C@H](CC[C@@H]5O[C@H]8[C@H]7O6)CC(=O)C[C@@H]2[C@H]1OC)CC4=C)C[C@@H](C)C3=C]

15. The antibody-drug conjugate according to claim 14, wherein,
Ab is an antibody or antigen-binding fragment; preferably an anti-ErbB2/HER2 antibody (for example, Trastuzumab);
(P)ₜ-L- is selected from:

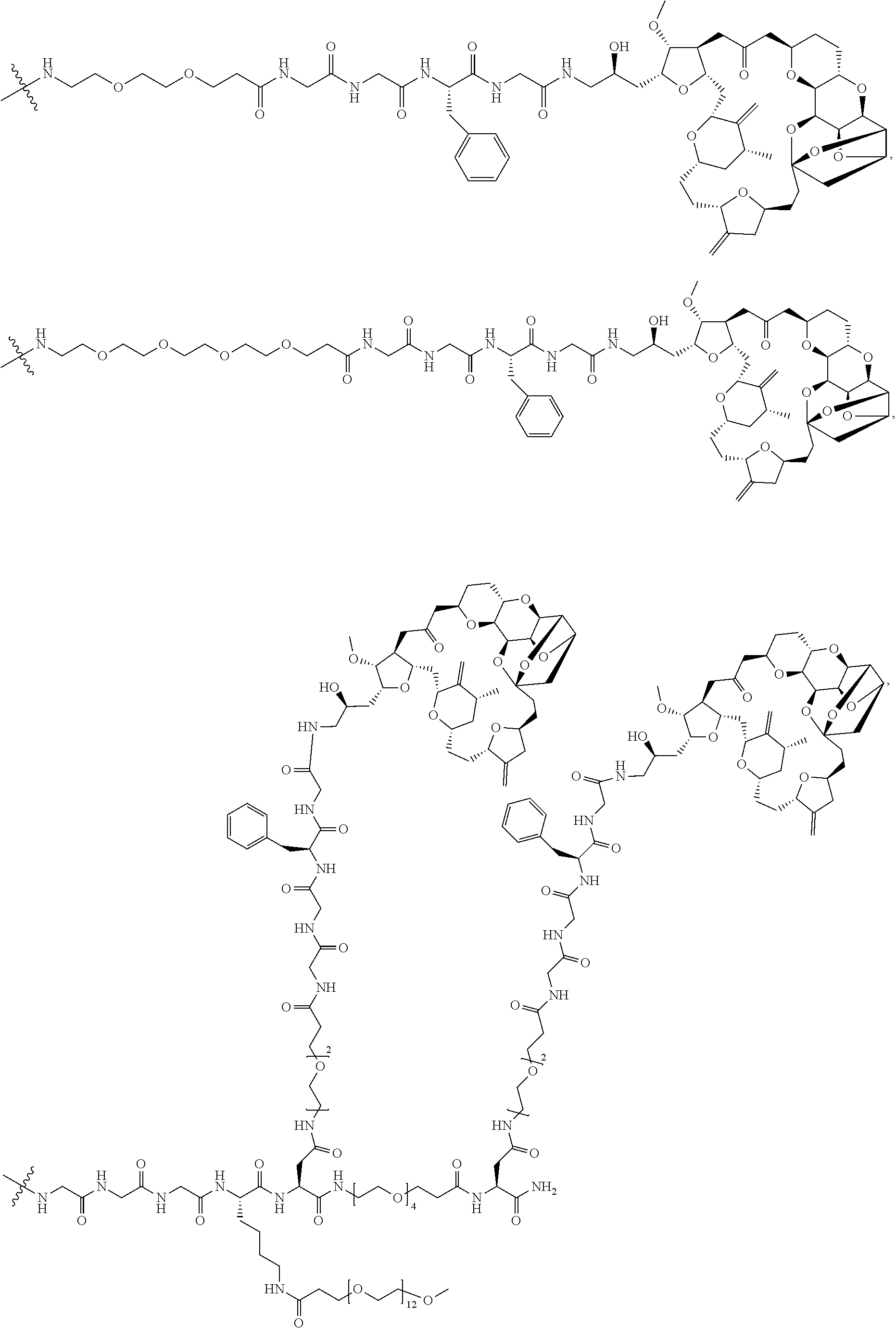

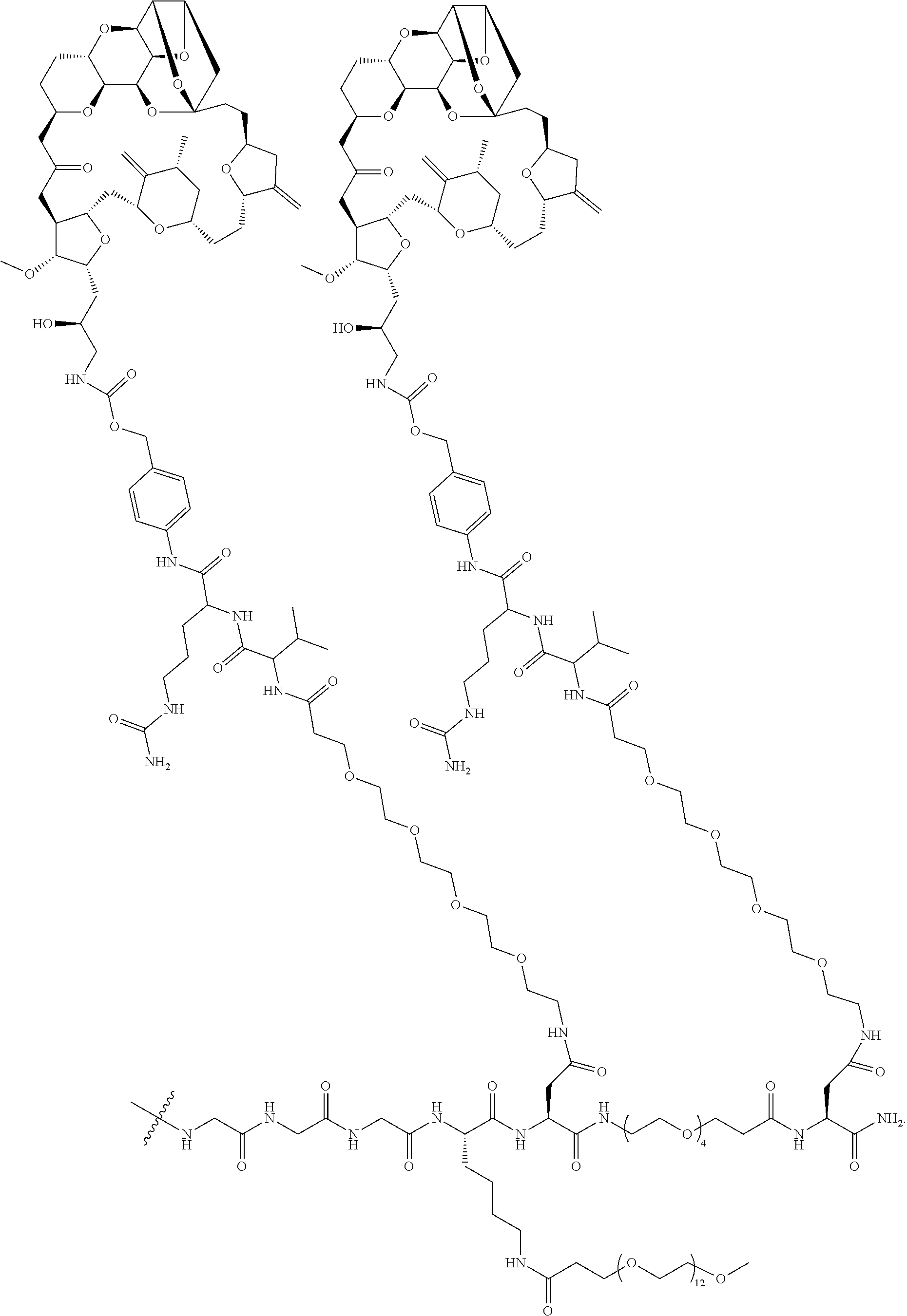

16. A method for preparing the antibody-drug conjugate according to claim 14, which comprises:
a) cleaving off the N-glycan chain part of the antibody Ab to afford a remodeled antibody of which the Fc region N-glycosylation site is N-acetylglucosamine or fucosyl-α-1,6-N-acetylglucosamine, under the catalysis of glycosidase or a mutant thereof;
b) conjugating the remodeled antibody with the linker-payload compound according to any one of claims 1 to 12 under the catalysis of glycosidase or a mutant thereof;
wherein, the glycosidase or the mutant thereof used in step a) and step b) is identical or different.

17. The method according to claim 16, wherein in steps a) and b), the glycosidase or a mutant thereof is fucose hydrolase, N-acetylglucosamine endohydrolase or a mutant thereof,
optionally wherein the N-acetylglucosamine endohydrolase comprises at least one of Endo-S (Streptococcus pyogenes endoglycosidase —S), Endo —F3 (Elizabeth Kingia miricola endoglycosidase-F3), Endo-S2(Endoglycosidase-S2, Streptococcus pyogenes endoglycosidase —S2), Endo-Sd(Endoglycosidase-Sd, Streptococcus pyogenes endoglycosidase-Sd) and Endo-CC (Endoglycosidase-CC, Streptococcus pyogenes endoglycosidase-CC); preferably, endoglycosidase is Endo H, Endo D, Endo F2, Endo F3, Endo M, Endo CC1, Endo CC2, Endo Om, Endo S and Endo S2.

18. (canceled)

19. An antibody-drug conjugate, which is prepared by the method according to claim 16.

20. A pharmaceutical composition, which comprises the antibody-drug conjugate according to claim 14.

21. (canceled)

22. A method for treating or preventing a tumor or an autoimmune disease, which comprises administering a therapeutically effective amount of the antibody-drug conjugate according to claim 14 to a subject in need thereof.
